(19) United States
(12) Patent Application Publication
(10) Pub. No.: US 2026/0234588 A1
(43) Pub. Date: Aug. 13, 2026

(54) TYPE II CAS PROTEIN AND USES THEREOF

(71) Applicant: GenEditBio Limited, Hong Kong (CN)

(72) Inventor: Bang WANG, Hong Kong (CN)

(21) Appl. No.: 19/632,884

(22) Filed: Mar. 30, 2026

Related U.S. Application Data
(63) Continuation of application No. PCT/CN2025/124486, filed on Sep. 26, 2025.

(30) Foreign Application Priority Data
Sep. 27, 2024 (WO) PCT/CN2024/121851

(51) Int. Cl. C12N 9/22; C12N 15/11; C12N 15/90

(52) U.S. Cl. CPC C12N 9/226; C12N 15/11; C12N 15/907; C12N 2310/20

(57) ABSTRACT

The current disclosure pertains to Type II Cas proteins, CRISPR-Cas systems, and their various applications. The Type II Cas proteins outlined in this disclosure broaden the utility of CRISPR-Cas systems for targeting or modifying genes.

This application is a continuation of International Application No. PCT/CN2025/124486, filed on 26 Sep. 2025, which claims the priority to provisional patent application PCT/CN2024/121851, filed on 27 Sep. 2024. The entire contents of these applications are hereby incorporated by reference.

### REFERENCE TO AN ELECTRONIC SEQUENCE LISTING

The contents of the electronic sequence listing (The Sequence Listing.xml; Size: 1,598,000 bytes; and Date of Creation: Mar. 27, 2026) is herein incorporated by reference in its entirety.

### TECHNICAL FIELD

The present disclosure relates to a Type II Cas protein and a CRISPR-Cas system for gene targeting and editing.

### BACKGROUND OF DISCLOSURE

Among the various CRISPR-Cas systems explored, the CRISPR-Cas9 system, part of the class 2 CRISPR-Cas systems, has been particularly influential in genome editing and holds significant potential for biomedical research. Despite its versatility, the CRISPR-Cas9 system faces challenges such as a limited number of known orthologs, restricted genomic targeting scope, and relatively modest editing efficiency. Considering the vast diversity of microbial genomes, it is likely that numerous CRISPR-Cas variants remain undiscovered, many of which may offer improved target recognition or higher editing efficiency compared to the currently available commercial versions.

### SUMMARY OF DISCLOSURE

The present disclosure is directed to meeting these needs. The discovery of novel Cas proteins enables the development of CRISPR-Cas systems with superior gene editing efficiency and/or specificity.

In one aspect, this disclosure provides an engineered, non-naturally occurring Type II CRISPR-associated (Cas) protein comprising: an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 1-77.

In some embodiment, this disclosure provides an engineered, non-naturally occurring Type II CRISPR-associated (Cas) protein comprising an amino acid sequence that has 100%, or at least 70%, at least 75%, at least 80%, at least 85%, at least 90%, at least 92%, at least 95%, at least 98%, or at least 99% sequence identity to any one of the amino acid sequence of SEQ ID NOs: 1-77, with the exception of the amino acid “M” at position 1 of the sequence.

In another aspect, this disclosure provides an engineered, non-naturally occurring Type II CRISPR-associated (Cas) protein comprising: an amino acid sequence that has 100%, or at least 70%, at least 75%, at least 80%, at least 85%, at least 90%, at least 92%, at least 95%, at least 98%, or at least 99% sequence identity to the amino acid sequence of SEQ ID NO: 69, and which comprises an amino acid mutation at one or more of the following positions: A1113, L1298, K1315 and Q1191.

In some embodiments, this disclosure provides an engineered, non-naturally occurring Type II CRISPR-associated (Cas) protein comprising: an amino acid sequence that: (i) has 100%, or at least 70%, at least 75%, at least 80%, at least 85%, at least 90%, at least 92%, at least 95%, at least 98%, or at least 99% sequence identity to the amino acid sequence of SEQ ID NO: 69, with the exception of the amino acid “M” at position 1 of the sequence; and (ii) comprises an amino acid mutation at one or more of the following positions: A1113, L1298, K1315 and Q1191.

In some embodiments, the Cas protein comprises an amino acid mutation at one or more of the following positions: A1113, L1298, K1315 and Q1191. In some embodiments, the Cas protein comprises an amino acid mutation at A1113, and optionally mutations at one or more of the following positions: L1298, K1315 and Q1191. In some embodiments, the Cas protein comprises an amino acid mutation at L1298, and optionally mutations at one or more of the following positions: A1113, K1315 and Q1191. In some embodiments, the Cas protein comprises an amino acid mutation at K1315, and optionally mutations at one or more of the following positions: A1113, L1298 and Q1191. In some embodiments, the Cas protein comprises an amino acid mutation at Q1191, and optionally mutations at one or more of the following positions: A1113, L1298 and K1315. In some embodiments, the Cas protein comprises an amino acid mutation at Q1191 and A1113, and optionally mutations at one or more of the following positions: L1298 and K1315. In some embodiments, the Cas protein comprises an amino acid mutation at Q1191 and L1298, and optionally mutations at one or more of the following positions: A1113 and K1315. In some embodiments, the Cas protein comprises an amino acid mutation at Q1191 and K1315, and optionally mutations at one or more of the following positions: A1113 and L1298. In some embodiments, the Cas protein comprises an amino acid mutation at A1113 and L1298, and optionally mutations at one or more of the following positions: Q1191 and K1315. In some embodiments, the Cas protein comprises an amino acid mutation at A1113 and K1315, and optionally mutations at one or more of the following positions: Q1191 and L1298. In some embodiments, the Cas protein comprises an amino acid mutation at L1298 and K1315, and optionally mutations at one or more of the following positions: Q1191 and A1113. In some embodiments, the Cas protein comprises an amino acid mutation at the following mutations: A1113, L1298 and K1315. In some embodiments, the mutation at position K1315 is selected from any one of K1315Q, K1315N and K1315T. In some embodiments, the mutation at position A1113 is selected from any one of A1113N, A1113K and A1113R. In some embodiments, the mutation at position Q1191 is selected from any one of Q1191N, Q1191K and Q1191V. In some embodiments, the mutation at position L1298 is L1298R. In some embodiments, the Cas protein comprises one or more of the following mutations: A1113R and K1315Q. In some embodiments, the Cas protein comprises one or more of the following mutations: L1298R and K1315Q. In some embodiments, the Cas protein comprises one or more of the following mutations: A1113R and K1315T. In some embodiments, the Cas protein comprises one or more of the following mutations: A1113R, L1298R and K1315Q. In some embodiments, the Cas protein comprises one or more of the following mutations: A1113R, L1298R and K1315T.

In some embodiments, the Cas protein is capable of recognizing a protospacer adjacent motif (PAM) having a sequence selected from the group consisting of: NATACT, NATAGT, NATAAT and NATATT. In some embodiments, the Cas protein comprises an amino acid mutation at K1315, and is capable of recognizing a protospacer adjacent motif (PAM) having a sequence selected from the group consisting of: NATACT, NATAGT, NATAAT and NATATT; optionally, the mutation at position K1315 is K1315Q or K1315T.

In some embodiments, the Cas protein comprises an amino acid mutation at K1315Q.

In some embodiments, the Cas protein comprises an amino acid mutation at A1113 and exhibits a higher activity when compared to that of the wild type Cas protein sequence: SEQ ID NO: 69.

In some embodiments, the mutation at position A1113 is A1113R.

In some embodiments, the Cas protein comprises an amino acid mutation at L1298 and exhibits a higher activity when compared to that of the wild type Cas protein sequence: SEQ ID NO: 69.

In some embodiments, the mutation at position L1298 is L1298R.

In some embodiments, the Cas protein comprises amino acid mutations at A1113 and L1298, and wherein the Cas protein exhibits a higher activity when compared to that of the wild type Cas protein sequence: SEQ ID NO: 69; optionally, the mutation at L1298 is L1298R and/or the mutation at A1113 is A1113R. In some embodiments, the Cas protein comprises amino acid mutations at A1113 and K1315; and wherein the Cas protein exhibits a higher activity when compared to that of the wild type Cas protein sequence: SEQ ID NO: 1 or 61; and wherein the Cas protein is capable of recognizing a protospacer adjacent motif (PAM) having a sequence selected from the group consisting of: NATACT, NATAGT, NATAAT and NATATT.

In some embodiments, the mutation at position K1315 is K1315Q or K1315T, and/or the mutation at position A1113 is A1113R.

In some embodiments, the Cas protein comprises amino acid mutations at K1315 and L1298; and wherein the Cas protein exhibits a higher activity when compared to that of the wild type Cas protein sequence: SEQ ID NO: 69; and wherein the Cas protein is capable of recognizing a protospacer adjacent motif (PAM) having a sequence selected from the group consisting of: NATACT, NATAGT, NATAAT and NATATT.

In some embodiments, the mutation at position K1315 is K1315Q or K1315T, and/or the mutation at position L1298 is L1298R.

In some embodiments, the Cas protein comprises amino acid mutations at A1113, K1315 and L1298; and wherein the Cas protein exhibits a higher activity when compared to that of the wild type Cas protein sequence: SEQ ID NO: 69; and wherein the Cas protein is capable of recognizing a protospacer adjacent motif (PAM) having a sequence selected from the group consisting of: NATACT, NATAGT, NATAAT and NATATT.

In some embodiments, the mutation at position K1315 is K1315Q or K1315T, the mutation at position A1113 is A1113R, and/or the mutation at position L1298 is L1298R.

In some embodiments, the Cas protein is a nickase or a dead Cas protein.

In some embodiments, the Cas protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 81-83; with an amino acid mutation at one or more of the following positions corresponding to: A1113, L1298 and Q1191 of SEQ ID NO: 69.

In some embodiments, the Cas protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 84-86; with an amino acid mutation at one or more of the following positions corresponding to: K1315, L1298 and Q1191 of SEQ ID NO: 69.

In some embodiments, the Cas protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 87-89; with an amino acid mutation at one or more of the following positions corresponding to: A1113, K1315, and L1298 of SEQ ID NO: 69.

In some embodiments, the Cas protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 90; with an amino acid mutation at one or more of the following positions corresponding to: A1113, K1315 and Q1191 of SEQ ID NO: 69.

In some embodiments, the Cas protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 81-95.

In some embodiments, the Cas protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 81-95, with an amino acid mutation at one or more of the following positions corresponding to: A1113, L1298, K1315 and Q1191 of SEQ ID NO: 69.

In some other aspects, this disclosure also provides a fusion protein comprising the engineered, non-naturally occurring Type II Cas protein as disclosed herein.

In some embodiments, the fusion protein further comprises a fusion partner. In some embodiment, the sequence of the fusion partner selected from the group consisting of: a nuclear localization signal sequence, a nuclear export signal sequence, a cell-penetrating peptide sequence, an affinity tag sequence, a deaminase sequence, a reverse transcriptase sequence, a recombinase sequence, a methyltransferase sequence, a methylase sequence, an acetylase sequence, an acetyltransferase sequence, a transcriptional activator sequence, a transcriptional repressor domain sequence, a cryptochrome sequence, a light inducible/controllable domain sequence, and a chemically inducible/controllable domain sequence.

In some embodiments, the fusion protein further comprises one or more of a nuclear localization signal sequence, a nuclear export signal sequence, a cell penetrating peptide sequence, or an affinity tag. In some embodiment, the fusion protein comprises one or more nuclear localization signal(s) NLS(s). The NLS(s) can locate at the end the Cas protein. The NLS(s) located each end or other portion of the Cas9 amino acid sequence can be same or not. In some embodiments, the NLS of the N-terminal end and the NLS of the C-terminal end are the same. In some embodiments, the NLS of the N-terminal end and the NLS of the C-terminal end are different. In some embodiments, the N-terminal end of the Cas protein sequence comprising one NLS and the C-terminal end of the Cas protein sequence comprising one NLS. The amino acid sequence of NLS fused to the N-terminal end and/or the C-terminal end of the Cas protein sequence respectively. NLS maybe an SV40 (simian virus 40) NLS, c-Myc NLS, or other suitable monopartite NLS. The NLS may be fused to an N-terminal and/or a C-terminal of the Cas protein. In some embodiments, an affinity tag (such as GST, FLAG or hexahistidine sequences) is utilized for purification of the Cas protein by affinity chromatography. In some embodiments, the amino acid sequence of the C-terminal FLAG sequence. Other available sequences and different combinations can also be chosen for the NLSs sequences and FLAG sequence.

In some embodiments, the fusion protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 521-597, 589.

In some embodiments, the fusion protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 711-725.

In some embodiments, the fusion protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 711-725, with an amino acid mutation at one or more of the positions corresponding to: A1113, L1298, K1315 and Q1191 of SEQ ID NO. 69.

In some embodiments, the Cas protein comprises an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequence of SEQ ID NOs: 81-95, 711-725, with the exception of the amino acid “M” at position 1 of the sequence; and with an amino acid mutation at one or more of the following positions: A1113, L1298, K1315 and Q1191.

In some embodiments, the Cas protein is a nickase or dead Cas protein. The DNA cleavage domain of an active Cas protein in this disclosure include two subdomains, the HNH nuclease subdomain and the RuvC subdomain. Mutations within these subdomains can silence the nuclease activity of the Cas protein.

This disclosure also provides an engineered, non-naturally occurring CRISPR-Cas system comprising:
(a) (i) the Cas protein or the fusion protein described herein; or (ii) the polynucleotide encoding the Cas protein or the fusion protein thereof; and
(b) at least one engineered guide RNA (gRNA) or at least one engineered polynucleotide encoding the gRNA thereof, wherein said gRNA comprises a spacer sequence that is complementary to a target nucleic acid and a Cas protein binding segment that interacts with said Cas protein or said Cas protein portion of the fusion protein, wherein the Cas protein binding segment comprises a tracrRNA sequence and a direct repeat (DR) sequence, and wherein the tracrRNA sequence hybridizes with the DR sequence to form a double-stranded RNA (dsRNA) duplex.

In some embodiments, the guide RNA is a dual guide RNA.

In some embodiments, the gRNA further comprises a linker sequence connecting the tracrRNA sequence and the DR sequence to form a sgRNA scaffold. In some typical embodiments, the linker comprises a short sequence of GAAA. In some embodiments, the linker serves as an artificial loop. In some embodiments, the sgRNA comprises, in an arrangement: (a) a spacer sequence, which is capable of hybridizing to a sequence of the target nucleic acid to be manipulated; (b) a DR sequence; (c) a linker sequence and (d) tracrRNA sequence. The tandem arrangement of the spacer sequence, the DR sequence, the linker sequence and tracrRNA sequence is in a 5′ to 3′ orientation, or in a 3′ to 5′orientation;

In some embodiments, the tracrRNA sequence is modified to lead the system having an enhancing gene editing activity when compared to that with wild type sequence (SEQ ID NO: 821). In some embodiments, the tracrRNA sequence is modified to enhance the stability of the gRNA when compared to that with wild type sequence (SEQ ID NO: 821).

In some embodiments, the tracrRNA sequence is modified to reduce the interaction between the spacer sequence and the tracrRNA sequence, optionally, the modification of the tracrRNA sequence comprises the group consisting of: one or more nucleotides mutation, one or more nucleotides insertion, one or more nucleotides deletion.

In some embodiments, the tracrRNA is modified to make the sequence comprise at least one stabilized hairpin secondary structure at a position that does not interfere with oligonucleotide binding and/or editing. In some embodiments, (i) the stabilized hairpin forms a secondary structure comprising a contiguous stem having a length of 1, 2, 3, 4 or 5 nt more than that of the wild type sequence (SEQ ID NO: 821), and/or (ii) the stabilized hairpin forms a secondary structure comprising a contiguous stem having 1 C-G base pairs, at least 2 C-G base pairs, at least 3 C-G base pairs, at least 4 C-G base pairs, or at least 5 C-G base pairs more than that of the wild type sequence (SEQ ID NO: 821).

In some embodiments, the tracrRNA sequence comprises a sequence having 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 821-826.

In some embodiments, the sgRNA scaffold comprises a sequence having 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 841-846.

In some embodiments, the spacer sequence hybridizes to one or more nucleic acid in a prokaryotic cell or in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the eukaryotic cell comprises a mammalian cell. In some embodiments, the mammalian cell comprises a human cell. In some embodiments, the eukaryotic cell comprises a plant cell.

In some embodiments, the polynucleotide encoding the Cas protein or the fusion protein is operably linked to a promoter; optionally, the promoter is a constitutive promoter, a tissue-specific promoter, or an inducible promoter. In some embodiments, the polynucleotide encoding the Cas protein or the fusion protein is operably linked to a promoter and is present in a vector; optionally, the vector is selected from the group consisting of: a retroviral vector, a lentiviral vector, a phage vector, an adenoviral vector, an adeno-associated virus vector, a herpes simplex virus vector and a plasmid vector.

In some embodiments, the system further comprising a donor template nucleic acid.

In another aspect, this disclosure also provides a gRNA comprising the features as defined above.

In another aspect, this disclosure also provides an engineered, non-naturally occurring polynucleotide encoding the Cas protein herein or the fusion protein herein.

In some embodiments, the polynucleotide encoding the Cas protein or the fusion protein is operably linked to a promoter and is presented in a vector; optionally, the vector is selected from the group consisting of: a retroviral vector, a lentiviral vector, a phage vector, an adenoviral vector, an adeno-associated virus vector, a herpes simplex virus vector and a plasmid vector.

In some embodiments, the polynucleotide is a ribonucleotide sequence or a deoxyribonucleotide sequence, or analogs thereof; optionally, the polynucleotide is codon-optimized for expression in a cell of interest; In some embodiments, the polynucleotide is codon optimized for expression in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell, preferably a human cell. In some embodiments, the cell is a mammalian cell, preferably a human cell.

In some embodiments, the polynucleotide is an mRNA and further comprises a 5′ cap sequence and/or a poly-A tail sequence. In some embodiments of the present disclosure, the mRNA utilized may be modified to enhance its functional properties and stability. Specifically, in some embodiments, the modification process involves the substitution of uridine (represented by the letter “U”) with N1-Methylpseudouridine or pseudouridine. This substitution is designed to improve the mRNA's resistance to degradation by ribonucleases, potentially increasing its half-life and translational efficiency within the cell. The incorporation of N1-Methylpseudouridine or pseudouridine into the mRNA structure can also positively influence the immune response profile, as these modifications have been shown to reduce the immunogenicity of mRNA molecules when compared to their unmodified counterparts. This is particularly crucial for the development of mRNA-based therapeutics and vaccines, where minimizing adverse immune reactions is paramount.

In some embodiments, the polynucleotide in this disclosure is codon-optimized for expression in a eukaryotic cell; optionally, the eukaryotic cell is selected from the group consisting of: a plant cell, a fungal cell, a single-cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

This disclosure also provides an engineered vector comprising the polynucleotide described herein.

In some embodiments, the vector is an expression vector. In some embodiments, the vector is an inducible, conditional, or constitutive expression vector. In some embodiments, (i) the polynucleotide encoding the Cas protein or the fusion protein, and (ii) the polynucleotides encoding the guide RNA are on a same vector. In some embodiments, (i) the polynucleotide encoding the Cas protein or the fusion protein, and (ii) the polynucleotides encoding the guide RNA are on different vectors.

This disclosure also provides a vector system comprising (a) one or more polynucleotides encoding the Cas protein or the fusion protein described herein; and (b) one or more polynucleotides encoding a guide RNA; wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence, wherein the tracrRNA sequence hybridizes with the DR sequence to form a double-stranded RNA (dsRNA) duplex.

In some embodiments, (i) the polynucleotide encoding the Cas protein or the fusion protein, and (ii) the polynucleotides encoding the guide RNA are on a same vector or on different vectors.

This disclosure also provides an engineered, non-naturally occurring cell comprising: (a) the Cas protein or fusion protein described herein; (b) the polynucleotide encoding said Cas protein or said fusion protein of (a); (c) the CRISPR-Cas system described herein, (d) the vector described herein; or (e) the vector system described herein.

This disclosure also provides a cell modified by utilizing: (a) the Cas protein or fusion protein described herein; (b) the polynucleotide encoding said Cas protein or said fusion protein of (a); (c) the CRISPR-Cas system described herein, (d) the vector described herein; or (e) the vector system described herein.

In some embodiments, the cell is an isolated eukaryotic cell wherein a target locus of interest is modified.

In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In some embodiments, the cell is a vertebrate, mammalian, rodent, goat, pig, bird, chicken, turkey, cow, horse, sheep, fish, primate, or human cell. In some embodiments, the cell is a mammalian cell. In some embodiments, the cell is a human cell. In some embodiments, the cell is a somatic cell, a germ cell, or a prenatal cell. In some embodiments, the cell is a zygotic cell, a blastocyst cell, an embryonic cell, a stem cell, a mitotically competent cell, or a meiotically competent cell. In some embodiments, the cell is not part of a human embryo. In some embodiments, the cell is a somatic cell. In one embodiment, the cell is a T cell, a CD8+ T cell, a CD8+ naive T cell, a central memory T cell, an effector memory T cell, a CD4+ T cell, a stem cell memory T cell, a helper T cell, a regulatory T cell, a cytotoxic T cell, a natural killer T cell, a Hematopoietic Stem Cell, a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In some embodiments, the cell is a T cell, a Hematopoietic Stem Cell, a retinal cell, a cochlear hair cell, a pulmonary epithelial cell, a muscle cell, a neuron, a mesenchymal stem cell, an induced pluripotent stem (iPS) cell, or an embryonic stem cell. In some other embodiments, the cell is a plant cell.

In some embodiments, said modifying of the target locus comprises inducing a DNA strand break. In some embodiments, said modifying a target locus comprises inducing a DNA double strand break or a DNA single strand break. In some embodiments, said modifying a target locus comprises altering gene expression of one or more genes. In some embodiments, said modifying a target locus comprises epigenetic modification of said target DNA locus.

In another aspect, this disclosure also provides a kit comprising: the Cas protein or fusion protein described herein, the polynucleotide described herein, the CRISPR-Cas system described herein, the vector described herein, the vector system described herein, or the cell described herein.

The kits described in this disclosure may encompass one or more containers with components essential for performing the methods in this disclosure, and may optionally contain instructions for use. Any of the kits delineated may additionally comprise ancillary components required for the execution of the editing methods. Each component within the kits, where applicable, may be provided in a liquid form (e.g., dissolved in solution) or in a solid form (e.g., lyophilized powder). In specific embodiments, some components may be reconstituted or otherwise processed (e.g., to an active state) upon the addition of a suitable solvent or other substance (such as water or buffer), which may or may not be furnished with the kit. In some embodiments, the kit may further comprise other suitable excipients such as buffers or reagents for facilitating the application of the kit. Preferably, the kit may be applied in various applications such as medical applications including therapies and diagnosis, researches and the like. Accordingly, the kit of the present disclosure may be used in the preparation of a medicament for treatment and/or in the preparation of an agent for research study.

The Cas protein described herein, fusion protein described herein, CRISPR-Cas system described herein, polynucleotide described herein, can be delivered by various delivery systems such as vectors, e.g., plasmids, viral delivery vectors, such as adeno-associated viruses (AAV), lentiviruses, adenoviruses, and other viral vectors, or methods, such as nucleofection or electroporation of ribonucleoprotein complexes consisting of Type V-I effectors and their cognate RNA guide or guides. The proteins and one or more RNA guides can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In some other aspects, this disclosure also provides a pharmaceutical composition comprising: (a) the Cas protein described herein; (b) the fusion protein described herein; (c) the polynucleotide described herein; (d) the CRISPR-Cas system described herein; (e) the vector described herein; (f) the vector system described herein; or (g) the cell described herein.

In some embodiments, the pharmaceutical composition further comprises a delivery system selected from: AAV (adeno-associated viruses), Adenoviruses, retroviruses, HSV (herpes simplex virus), Gammaretrovirus, LV (lentivirus), eCIS (extracellular Contractile Injection System), VLPs (virus-like particles), liposomes, plasmid, LNPs (lipid nanoparticles), exosomes, microvesicles, nucleic acid nanoassemblies, a gene gun, and an implantable device.

In some other aspects, this disclosure also provides the use of the Cas protein described herein; the fusion described herein the polynucleotide described herein, the CRISPR-Cas system described herein, the vector described herein, the vector system described herein, the cell described herein, the kit described herein, or the pharmaceutical composition described herein for the treatment, prevention, diagnosis, or detection of a disease.

In some other aspects, this disclosure also provides a method of modifying or targeting a target DNA locus, wherein the method comprising delivering to said locus: the Cas protein described herein; the fusion protein described herein; the polynucleotide described herein; the CRISPR-Cas system described herein; the vector described herein; the vector system described herein; the kit described herein or the pharmaceutical composition described herein.

In some embodiments, said modifying or targeting a target locus comprises inducing a DNA strand break. In some embodiments, said modifying or targeting a target locus comprises inducing a DNA double strand break or a DNA single strand break. In some embodiments, said modifying or targeting a target locus comprises altering gene expression of one or more genes. In some embodiments, said modifying or targeting a target locus comprises epigenetic modification of said target DNA locus. In some embodiments, the method is a method of modifying a cell, a cell line, or an organism by manipulation of one or more target sequences at genomic loci of interest.

In some other aspects, this disclosure also provides a method of cleaving a target DNA, the method comprising: contacting the target DNA with the Cas protein described herein; the polynucleotide described herein; the CRISPR-Cas system described herein; the vector described herein; the vector system described herein; the kit described herein or the pharmaceutical composition described herein.

In some embodiments, cleaving the target DNA sequence results in the formation of an indel or the insertion of a nucleotide sequence. In some embodiments, cleaving the target DNA or target nucleotide comprising cleaving the target DNA or target sequence in two sites, and results in the deletion or inversion of a sequence between the two sites. In some embodiments, the target DNA is a double stranded DNA or a single stranded DNA, or DNA-RNA hybrids.

In some embodiments, said modifying or targeting a target locus comprises inducing a DNA strand break, altering gene expression of one or more genes, or epigenetic modification of said target DNA locus; optionally, the DNA strand break comprise a DNA double strand break or a DNA single strand break.

In some embodiments, the method is performed ex vivo or in vivo.

This disclosure also provides an isolated eukaryotic cell comprising a modified target locus of interest, wherein the target locus of interest has been modified according to the method described in this disclosure.

This disclosure also provides a system for detecting the presence of a nucleic acid target sequence in an in vitro sample, comprising:
(a) a Cas protein described in this disclosure;
(b) at least one guide polynucleotide comprising a spacer sequence capable of binding the target sequence, and designed to form a complex with the Cas protein; and
(c) a nucleic acid-based masking construct comprising a non-target sequence; wherein the Cas protein exhibits collateral cleavage activity of RNA and/or ssDNA and cleaves the non-target sequence of the nucleic acid-based masking construct activated by the target sequence.

This disclosure also provides a method for detecting target nucleic acids in samples comprising: (a) contacting one or more samples with
(i) a Cas protein described in this disclosure;
(ii) at least one guide polynucleotide comprising a guide sequence designed to have a degree of complementarity with the target sequence, and designed to form a complex with the Cas protein; and
(iii) a nucleic acid-based masking construct comprising a non-target sequence; wherein the Cas protein exhibits collateral cleavage activity of RNA and/or ssDNA and cleaves the non-target sequence of the nucleic acid-based masking construct activated by the target sequences; and
(b) detecting a signal from cleavage of the non-target sequence, thereby detecting the one or more target sequences in the sample.

These and other aspects, objects, features, and advantages of the example embodiments will become apparent to those having ordinary skill in the art upon consideration of the following detailed description of illustrated example embodiments.

### BRIEF DESCRIPTION OF FIGURES

FIG. 1 shows the GEBx0530 RNP structure.

FIG. 2A and FIG. 2B show the PAM preference of Cas proteins in HEK293 cell line.

FIG. 3 shows the in vitro gene editing activity and spacer length preference of GEBx0530 in HEK293 cell line.

FIG. 4 shows a rendering of the predicted RNP structure of GEBx0530 (A), highlighting the amino acid side chains adjacent to the GGTACT PAM (B).

FIG. 5 shows the PAM preference of the wild type GEBx0530 in HEK293 cell line.

FIG. 6A and FIG. 6B illustrate the PAM preference of the GEBx0530 single mutant variants in the HEK293 cell line.

FIG. 7 presents the indel activity of human HEK293T cells following reverse transfection with the pGEBx0530-WT plasmid and corresponding psgRNA plasmid.

FIG. 8 illustrates the PAM preference of the GEBx0530 double mutant variants in the HEK293 cell line.

FIG. 9 presents a mean nuclease activity plots for GEBx0530-WT and GEBx0530-DM3 on 24 sites with NATANT PAMs in human cells. The central black line represents the mean of 6 sites for each PAM class.

FIG. 10 presents the indel activity of human HEK293T cells following reverse transfection with the pGEBx0530-WT plasmid and psgRNA plasmid that harbors different types of sgRNA truncations.

FIG. 11 illustrates the PAM preference of the GEBx0530 triple mutant variants in the HEK293 cell line.

FIG. 12 presents the indel activity of Hepa1-6 cells following transfection with VLPs harbors GEBx0530-TM-1 mutant and sgRNA targeted mouse TTR locus.

### DETAILED DESCRIPTION

The following examples further illustrate the present disclosure, but the present disclosure is not limited thereto.

It must be noted that as used herein and in the appended claims, the singular forms or the terms “a”, “an”, “the”, and “said” and similar terms used in the context of the present disclosure (especially in the context of the claims) are to be construed to cover both the singular and plural unless otherwise indicated herein or clearly contradicted by the context. In some embodiments, the above terms can be reasonably comprehended as “one” or “one or more”. Further, unless otherwise required by context, singular terms shall include pluralities and plural terms shall include the singular.

Unless otherwise indicated, it is intended that all singular/plural terms also encompass the active tense and past tense forms of a term, and it needs to be understood according to the context in the article.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and those terms such as “consisting essentially of” and “consists essentially of” have the meaning ascribed to them in U.S. Patent law. The term “a group consisting of” and the like refers to a specific set or collection of elements, components, or features. It may include one or more of the specified elements, components, or features. For example, a group consisting of: A, B, or C may refer to a set that includes any one or more of the specified elements A, B, or C. The claim encompasses the possibility of having any single element (A, B, or C) individually, any two elements combined (A and B, A and C, or B and C), or all three elements together (A, B, and C). This phrase defines the disclosure in terms of its variability within the specified options, allowing for different combinations of the listed elements while still maintaining the claimed scope.

When “t” or “T” appears in a sequence in this disclosure as a nucleotide of an RNA sequence, it should be understood as “u” or “U”.

The term “identity” in the context of two or more nucleic acids or polypeptide sequences refers to two or more sequences or subsequences that are the same or have a specified percentage of amino acid residues or nucleotides that are the same as measured using a BLAST or BLAST 2.0 or FASTA etc. sequence comparison algorithms with default parameters described below.

The term “exemplary” is used herein to mean serving as an example, instance, or illustration. Any aspect or design described herein as “exemplary” is not necessarily to be construed as preferred or advantageous over other aspects, embodiments, or designs.

As used in this disclosure, the term “optional” or “optionally” means that the subsequent described event, circumstance or substituent may or may not occur, and that the description includes instances where the event or circumstance occurs and instances where it does not.

The use of “or” or “/” is inclusive and means “and/or” unless stated otherwise; or it could be interpreted differently based on the context. The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “about”, “~” as used herein when referring to a measurable value such as a parameter, an amount, a temporal duration, and the like, are meant to encompass variations of and from the specified value It is to be understood that the value to which the modifier “about” or “~” refers is itself also specifically, and preferably, disclosed.

The term “exemplary” is used herein to mean serving as an example, instance, or illustration. Any aspect or design described herein as “exemplary” is not necessarily to be construed as preferred or advantageous over other aspects, embodiments, or designs.

“Encoding” refers to the property of specific sequences of nucleotides in a gene, such as a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. A polynucleotide encoding a protein includes all nucleotide sequences that are degenerate versions of each other and that code for the same amino acid sequence or amino acid sequences of substantially similar form and function.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. In all aspects and embodiments, whether they include these terms or not, it will be understood that, preferably, may be optional and thus preferably included or not preferably included. Furthermore, the terms “non-naturally occurring” and “engineered” may be used interchangeably and so can therefore be used alone or in combination and one or other may replace mention of both together. In particular, “engineered” is preferred in place of “non-naturally occurring” or “non-naturally occurring and/or engineered” or “engineered, non-naturally occurring”.

As presented in this disclosure, the term “cleavage event” as used herein, refers to a DNA break in a target nucleic acid created by a type II Cas nuclease of a CRISPR system described herein. In some embodiments, the cleavage event is a double-stranded DNA break. In some embodiments, the cleavage event is a single-stranded DNA break.

As presented in this disclosure, the term “targeting” refers to the ability of a complex including a CRISPR-associated protein and an RNA guide, to preferentially or specifically bind to, e.g., hybridize to, a specific target nucleic acid compared to other nucleic acids that do not have the same or similar sequence as the target nucleic acid.

As presented in this disclosure, the term “GEBx” followed by a numerical suffix is utilized as a generic code to represent either nucleic acids or proteins. It is important to note that the use of identical codes for nucleic acids and proteins, or derivatives thereof, does not imply that the substances represented by these codes are identical. In other words, GEBx-ns (e.g. GEBx0530) may refer to a specific nucleic acid sequence in one instance and a distinct protein in another. Some embodiments may illustrate a direct correspondence between the nucleic acid and protein denoted by the same or derived codes. Thus, the code “GEBx” serves as an indexing system to organize and reference the diverse biomolecules described in this disclosure, and the meaning of the code will be understood based on the context provided.

Various embodiments are described in this disclosure. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “in some embodiment(s)”, “in certain embodiment(s)”, “in some preferred embodiments”, “in some typical embodiment(s)”, “in typical embodiment(s)” or similar expressions means that a particular feature, structure or characteristic described in connection with the embodiment is included in at least one embodiment of the present disclosure. Furthermore, a particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any one of the claimed embodiments can be used in any combination.

The recitation of numerical ranges by endpoints includes all numbers and fractions subsumed within the respective ranges, as well as the recited endpoints.

Definitions and explanations of terms provided in this disclosure are to be understood as applicable throughout this specification, even when introduced in the context of a single aspect or embodiment, unless the context expressly requires a different interpretation.

In one aspect, this disclosure provides an engineered, non-naturally occurring type II Cas protein, wherein the type II Cas protein comprises an amino acid sequence having has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-77, or a variant thereof.

In another aspect, the disclosure provides a type II Cas protein comprises an amino acid sequence having 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-77, with the exception of “M” at position 1 of the sequence.

As presented in this disclosure, the term “Cas protein” or “CRISPR-associated protein” or other similar terms refers to a class of CRISPR-associated proteins that are integral components of the CRISPR-Cas system. In some embodiments, the ‘Cas protein’ can represent a Type II CRISPR-associated (Cas) protein, either alone or in combination with other effector domains. These proteins can possess intrinsic nuclease activity, enabling them to cleave double-stranded DNA or RNA molecules in a sequence-specific manner guided by a complementary RNA molecule, such as Cas9 and Cas12, which have been extensively utilized for genome editing applications. Additionally, some Cas proteins may be engineered to retain only one of the two active sites required for double-stranded cleavage, resulting in nickase activity, allowing them to introduce single-strand breaks in target nucleic acid sequences for controlled cleavage events. Furthermore, certain Cas proteins may be modified to lack any inherent nuclease activity, referred to as dead Cas or nuclease-inactive Cas proteins. Despite the absence of enzymatic function, these dead Cas proteins maintain their ability to bind specifically to target nucleic acid sequences and are often used in conjunction with other effector domains (or functional domains) for applications such as gene regulation, epigenome editing, and as components of advanced imaging systems. In some embodiments, the Cas protein may be used to reduce off-target effects.

In some embodiments, the active Cas nuclease, nickase or dead Cas may also be part of a fusion protein containing another effector domain. The fusion proteins comprising such other effector domains (or functional domains) and such active Cas nuclease, nickase or dead Cas are also involved in the scope of the Cas protein. In some embodiments, the Cas protein may be a split form. In some embodiments, the Cas protein may also be an inducible Cas protein. In some embodiments, the type II Cas protein may be part of a self-inactivating system (SIN); In some embodiments, the type II Cas nuclease may also be part of a synergistic activator system (SAM) as defined herein elsewhere.

In some embodiments, the domain arrangement of Type II Cas protein the Cas protein contains a RuvC domain, BH (bridge helix) domain, REC domain, HNH domain, and/or a CTD (C-terminal domain). The RuvC domain is a critical catalytic site responsible for the cleavage of target DNA strands. It contains three split RuvC sub-domains, which are intricately folded to form the active site where DNA cleavage occurs. These sub-domains work in coordination to recognize and cleave the DNA at specific locations directed by the guide RNA. The BH domain, or bridge helix domain, serves as a structural link between the different domains of the Cas protein. It is characterized as an arginine-rich region comprising numbers of arginine amino acids. This abundance of arginine residues is crucial for interactions with the phosphate backbone of the target DNA strand. The arginine residues can form hydrogen bonds with the phosphate groups, aiding in the proper positioning and alignment of the target DNA for cleavage. The REC domain, or recognition lobe, is involved in the recognition of the target DNA sequence. It contributes to the specificity of the Cas protein by distinguishing between target and non-target sequences, ensuring that only the intended DNA segments are cleaved. The HNH domain is another catalytic site that works in conjunction with the RuvC domain to cleave the complementary strand of the target DNA. It is named after its characteristic histidine-asparagine-histidine sequence and is essential for the nuclease activity of the Cas protein. The CTD, or C-terminal domain, is often involved in interactions with other proteins or cellular structures, contributing to the localization and regulation of the Cas protein within the cell. It may also play a role in the stability and overall conformation of the Cas protein, ensuring that it remains functional and specific in its targeting. The complex domain arrangement allows the Type II Cas protein to carry out its precise and crucial function within the CRISPR system, making it an invaluable tool for genome editing and manipulation.

As used in this disclosure, the “M” referred to herein stands for the starting amino acid Methionine, which is typically the initiating point for protein synthesis in many proteins. Naturally occurring Cas proteins often begin with Methionine as the first amino acid in their sequence. However, when scientists engineer these proteins, such as by fusing them with a Nuclear Localization Signal (NLS) or other domains, this initial amino acid “M” might be replaced or altered to introduce new functionalities or characteristics.

In this disclosure, particular attention has been given to the flexibility and functionality of the engineered Cas protein. Except for the intentional modification of the starting amino acid, Methionine (M), at position 1, designed Cas protein exhibits a significant sequence identity—ranging from 70% to 100%—when compared to the reference sequences. This strategic alteration not only aligns with our goal of tailoring the protein's properties but also ensures that the core or expected functionalities intrinsic to the Cas protein are preserved. By manipulating the initial amino acid without compromising the overall sequence similarity, some specific attributes are enhanced, such as improving cellular localization or introducing other advantageous features, while maintaining the fundamental characteristics that make Cas proteins indispensable tools in genomic manipulation.

This disclosure also provides an engineered, non-naturally occurring Type II CRISPR-associated (Cas) protein comprising an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 69, with an amino acid mutation at one or more of the following positions: A1113, L1298, K1315 and Q1191.

As used in this disclosure, the phrase a “mutation at one or more of the following positions” [e.g., A1113, L1298, K1315, and Q1191] shall be construed to mean a mutation at one or more amino acid positions corresponding to the specifically enumerated positions in the reference amino acid sequence as set forth in SEQ ID NO: 69; The position numbering (e.g., 1113, 1298) is exclusively based on the amino acid sequence of the designated reference sequence (SEQ ID NO: 69); In some embodiments, when referring to a mutation in a variant protein (e.g., a protein having a certain percentage of sequence identity to SEQ ID NO: 69), the corresponding position in the variant sequence is identified by alignment with the full-length reference sequence using a standard sequence alignment algorithm (e.g., BLAST, ClustalW) under default parameters; This definition applies equally to proteins that are derived from the reference sequence but have been modified, including, but not limited to: (a) Fusion Proteins: In a fusion protein comprising the Cas protein and a heterologous polypeptide, the specified positions refer to the residues within the Cas protein portion that correspond to the enumerated positions in the reference SEQ ID NO: 69; or (b) Fragments/Deletions/Insertions: In truncated, fragmented, or circularly permuted variants, or variants containing internal amino acid insertions or deletions, the corresponding position is determined by the optimal sequence alignment with the full-length reference sequence.

In some embodiments, the Cas protein comprises an amino acid mutation at one or more of the following positions: A1113, L1298, K1315 and Q1191. In some embodiments, the Cas protein comprises an amino acid mutation at A1113, and optionally mutations at one or more of the following positions: L1298, K1315 and Q1191. In some embodiments, the Cas protein comprises an amino acid mutation at L1298, and optionally mutations at one or more of the following positions: A1113, K1315 and Q1191. In some embodiments, the Cas protein comprises an amino acid mutation at K1315, and optionally mutations at one or more of the following positions: A1113, L1298 and Q1191. In some embodiments, the Cas protein comprises an amino acid mutation at Q1191, and optionally mutations at one or more of the following positions: A1113, L1298 and K1315. In some embodiments, the Cas protein comprises an amino acid mutation at Q1191 and A1113, and optionally mutations at one or more of the following positions: L1298 and K1315. In some embodiments, the Cas protein comprises an amino acid mutation at Q1191 and L1298, and optionally mutations at one or more of the following positions: A1113 and K1315. In some embodiments, the Cas protein comprises an amino acid mutation at Q1191 and K1315, and optionally mutations at one or more of the following positions: A1113 and L1298. In some embodiments, the Cas protein comprises an amino acid mutation at A1113 and L1298, and optionally mutations at one or more of the following positions: Q1191 and K1315. In some embodiments, the Cas protein comprises an amino acid mutation at A1113 and K1315, and optionally mutations at one or more of the following positions: Q1191 and L1298. In some embodiments, the Cas protein comprises an amino acid mutation at L1298 and K1315, and optionally mutations at one or more of the following positions: Q1191 and A1113. In some embodiments, the Cas protein comprises an amino acid mutation at the following mutations: A1113, L1298 and K1315. In some embodiments, the mutation at position K1315 is selected from any one of K1315Q, K1315N and K1315T. In some embodiments, the mutation at position A1113 is selected from any one of A1113N, A1113K and A1113R. In some embodiments, the mutation at position Q1191 is selected from any one of Q1191N, Q1191K and Q1191V. In some embodiments, the mutation at position L1298 is L1298R. In some embodiments, the Cas protein comprises one or more of the following mutations: A1113R and K1315Q. In some embodiments, the Cas protein comprises one or more of the following mutations: L1298R and K1315Q. In some embodiments, the Cas protein comprises one or more of the following mutations: A1113R and K1315T. In some embodiments, the Cas protein comprises one or more of the following mutations: A1113R, L1298R and K1315Q. In some embodiments, the Cas protein comprises one or more of the following mutations: A1113R, L1298R and K1315T.

One of the key elements of the CRISPR-Cas system associated modification process is the Protospacer Adjacent Motif (PAM), a short DNA sequence immediately adjacent to the target DNA sequence. The PAM sequence is essential for Cas protein binding and cleavage activity, ensuring that only the intended DNA sequences are targeted for editing. The ability of the Cas protein to recognize these specific PAM sequences is due to its unique structural features and binding interactions with the DNA. Each PAM sequence provides a distinct motif that the Cas protein can identify, ensuring accurate and efficient targeting. For example, the NRRANH sequence may offer a specific arrangement of nucleotides that the Cas protein can bind to with high affinity. These diverse range of PAM sequences expands the potential applications of the CRISPR-Cas system. It enables researchers and scientists to target a wider variety of DNA sequences for editing, increasing the versatility and effectiveness of this powerful gene-editing tool. Additionally, understanding these PAM sequences can aid in the development of more advanced Cas proteins with even greater targeting capabilities, further advancing the field of gene editing and its potential benefits for research, medicine, and biotechnology. This specific recognition of these Cas proteins allows for greater flexibility in selecting target DNA sequences for editing. In some embodiments, the Cas protein is mutated to be able to recognized a diversity PAM sequence.

In some embodiments, the Cas protein is capable of recognizing a protospacer adjacent motif (PAM) having a sequence selected from the group consisting of: NATACT, NATAGT, NATAAT and NATATT. In some embodiments, the Cas protein comprises an amino acid mutation at K1315, and is capable of recognizing a protospacer adjacent motif (PAM) having a sequence selected from the group consisting of: NATACT, NATAGT, NATAAT and NATATT; optionally, the mutation at position K1315 is K1315Q or K1315T.

As presented in this disclosure, in the PAM sequences provided, N: Represents any one of the four standard DNA nucleotides: Adenine (A), Thymine (T), Cytosine (C), or Guanine (G). This code facilitates the inclusion of any nucleotide at a given position without the need to specify it individually; R: Stands specifically for purine nucleotides, either Adenine (A) or Guanine (G). Purines are critical in DNA structure and function, and this code simplifies the incorporation of these larger nucleotides into PAM sequences; H: Denotes any nucleotide except Guanine (G). It can therefore represent Adenine (A), Thymine (T), or Cytosine (C). This code is useful for excluding Guanine (G) in specific positions where its larger size may impact structural or functional aspects of the sequence. Y: Indicates pyrimidine nucleotides, being either Thymine (T) or Cytosine (C). Pyrimidines are often found in specific regions of DNA and RNA, and this code aids in their inclusion without specifying the exact nucleotide. W: Represents weak bases, which can be either Adenine (A) or Thymine (T). This distinction is biochemically relevant as Adenine and Thymine have similar properties in certain contexts, such as hydrogen bonding. V: Reflects any nucleotide except Thymine (T), thus including Adenine (A), Cytosine (C), or Guanine (G). This code assists in situations where Thymine is not preferred or needed due to its unique chemical properties among the pyrimidines. M: Represents either Adenine (A) or Cytosine (C).

As presented in this disclosure, the terms “recognized”, “recognizing”, or “recognition” in this context refers to the capability of the Cas protein to form a functional complex with a sgRNA at a DNA target site to which the sgRNA hybridizes (i.e. to which the spacer sequence of the sgRNA hybridizes) and being flanked by the PAM sequence, and wherein the Cas protein is capable of performing its natural function, i.e. DNA cleavage or DNA binding. In this context it is to be noted that such DNA cleavage precludes the type II Cas protein from being a catalytically inactive type II Cas nuclease. In the case of for instance an inactivated type II Cas nuclease (e.g. a dead type II Cas nuclease), a complex between the type II Cas nuclease, sgRNA and cognate target may nevertheless be formed if the required PAM sequence is present, but such does not result in DNA cleavage.

In some embodiments, the Cas protein comprises an amino acid mutation at K1315Q. In some embodiments, the Cas protein comprises an amino acid mutation at A1113 and exhibits a higher activity when compared to that of the wild type Cas protein sequence: SEQ ID NO: 69. In some embodiments, the mutation at position A1113 is A1113R. In some embodiments, the Cas protein comprises an amino acid mutation at L1298 and exhibits a higher activity when compared to that of the wild type Cas protein sequence: SEQ ID NO: 69. In some embodiments, the mutation at position L1298 is L1298R.

In some embodiments, the Cas protein comprises an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequences of SEQ ID NOs: 81-83; with an amino acid mutation at one or more of the following positions corresponding to: A1113, L1298 and Q1191 of SEQ ID NO: 69.

In some embodiments, the Cas protein comprises an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequences of SEQ ID NOs: 84-86; with an amino acid mutation at one or more of the following positions corresponding to: K1315, L1298 and Q1191 of SEQ ID NO: 69.

In some embodiments, the Cas protein comprises an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequences of SEQ ID NOs: 87-89; with an amino acid mutation at one or more of the following positions corresponding to: A1113, K1315, and L1298 of SEQ ID NO: 69.

In some embodiments, the Cas protein comprises an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequences of SEQ ID NOs: 90; with an amino acid mutation at one or more of the following positions corresponding to: A1113, K1315 and Q1191 of SEQ ID NO: 69.

In some embodiments, the Cas protein comprises an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequences of SEQ ID NOs: 81-95.

In some embodiments, the Cas protein comprises an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of the amino acid sequences of SEQ ID NOs: 81-95, with an amino acid mutation at one or more of the following positions corresponding to: A1113, L1298, K1315 and Q1191 of SEQ ID NO: 69.

In some other aspects, this disclosure also provides a fusion protein comprising the engineered, non-naturally occurring Type II Cas protein as disclosed herein. The ‘Cas protein’ can represent a Type II CRISPR-associated (Cas) protein, either alone (in an unfused state) or in combination with other effector domains. The term “combination” should be construed to encompass any functional association formed by genetic fusion or non-covalent means. To define the preferred embodiments with greater precision, the present non-provisional application further clarifies and details the concept of a “fusion protein.” The term “fusion protein” as used herein represents and particularizes a preferred form of the aforementioned Cas protein “in combination with other effector domains (a fusion partner)” Specifically, it refers to a chimeric polypeptide formed by the genetic fusion, via peptide bonds, of the engineered Cas protein to one or more heterologous functional domains (e.g., effector domains, nuclear localization signals, tags, etc.). Therefore, the explicit recitation and description of “fusion protein” should be regarded as a further elaboration, refinement, and supplementation of preferred embodiments of the broader concept of a “Cas protein in combination with other effector domains,” which was disclosed in the priority document. It is to be understood that this does not constitute a departure from or an introduction of new subject matter relative to the priority disclosure. A person skilled in the art would directly and unambiguously recognize that the concept of “combination” described in the priority document naturally encompasses a “fusion protein” formed by genetic fusion.

In some embodiments, the fusion protein further comprises a fusion partner (effector domain or functional domain).

As presented in this disclosure, the term “fusion partner” refers to a protein, peptide, or polypeptide that is genetically or chemically linked to a target protein (e.g., the engineered Type II Cas protein described herein). The fusion partner may confer additional functions, properties, or regulatory capabilities to the target protein, such as enhancing stability, altering subcellular localization, improving solubility, providing detection tags (e.g., fluorescent proteins), or introducing enzymatic activity.

Such fusion partner can have one or more types of enzymatic activities, including polymerase activity, ligase activity, reverse transcriptase activity, deaminase activity, replication activity, or proofreading activity; In some embodiment, the effector domains domain comprises a nuclease, a nickase, a deaminase, a reverse transcriptase, a recombinase, a methyltransferase, a methylase, an acetylase, an acetyltransferase, a transcriptional activator, a transcriptional repressor domain, a cryptochrome, a light inducible/controllable domain, or a chemically inducible/controllable domain.

In some embodiments, the fusion protein further comprises one or more of a nuclear localization signal sequence, a nuclear export signal sequence, a cell penetrating peptide sequence, an affinity tag. The fusion protein comprises one or more nuclear localization signal(s) NLS(s). The NLS(s) can locate at the end Cas protein or the fusion protein. The NLS(s) located each end of the Cas protein or fusion protein can be same or not. In some embodiments, the NLS of the N-terminal end and the NLS of the C-terminal end are the same. In some embodiments, the NLS of the N-terminal end and the NLS of the C-terminal end are different. In some embodiments, the N-terminal end of the Cas protein comprising one NLS and the C-terminal end of the Cas protein comprising one NLS. The amino acid sequence of NLS fused to the N-terminal end and/or the C-terminal end of the Cas protein respectively. NLS maybe an SV40 (simian virus 40) NLS, c-Myc NLS, or other suitable monopartite NLS. The NLS may be fused to an N-terminal and/or a C-terminal of the Cas protein. In some embodiments, an affinity tag (such as GST, FLAG or hexahistidine sequences) is utilized for purification of the Cas protein by affinity chromatography. In some embodiments, the amino acid sequence of the C-terminal FLAG sequence. Other available sequences and different combinations can also be chosen for the NLSs sequences and FLAG sequence.

In some embodiments, the fusion protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 521-597, 589.

In some embodiments, the fusion protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 711-725.

In some embodiments, the fusion protein comprises an amino acid sequence that has 100%, or at least 70%, at least 75%, at least 80%, at least 85%, at least 90%, at least 92%, at least 95%, at least 98%, or at least 99% sequence identity to any one of the amino acid sequences of SEQ ID NOs: 711-725, with an amino acid mutation at one or more of the positions corresponding to: A1113, L1298, K1315 and Q1191 of SEQ ID NO. 69.

In some embodiments, the Cas protein comprises an amino acid sequence that has 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequence of SEQ ID NOs: 711-725, with the exception of the amino acid “M” at position 1 of the sequence; and with an amino acid mutation at one or more of the following positions corresponding to: A1113, L1298, K1315 and Q1191 of SEQ ID NO: 69.

In some embodiments, the Cas protein is a nickase or dead Cas protein. The DNA cleavage domain of an active Cas protein in this disclosure include two subdomains, the HNH nuclease subdomain and the RuvC subdomain. Mutations within these subdomains can silence the nuclease activity of the Cas protein.

This disclosure also provides an engineered, non-naturally occurring CRISPR-Cas system comprising:
(a) (i) the Cas protein or the fusion protein described herein; or (ii) the polynucleotide encoding the Cas protein or the fusion protein thereof; and
(b) at least one engineered guide RNA (gRNA) or at least one engineered nucleic acid encoding the guide RNA thereof, wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence, wherein the tracrRNA sequence hybridizes with the DR sequence to form a double-stranded RNA (dsRNA) duplex.

As presented in this disclosure, the term “complementary” describes the ability of two nucleic acid strands to pair with each other through their bases. This complementarity can occur via perfect base-pairing, where each base along one strand forms a specific hydrogen-bonded pair with its complementary base on the opposite strand, following the standard Watson-Crick base pairing rules: adenine (A) pairs with thymine (T) or uracil (U), and cytosine (C) pairs with guanine (G). This perfect base-pairing allows for precise recognition and binding between the two strands. Additionally, complementarity can also involve imperfect base-pairing, which includes situations where mismatches, insertions, or deletions may lead to non-standard base pairing or reduced affinity between the strands. Despite these imperfections, the strands maintain enough complementarity to interact, albeit with potentially reduced specificity or stability in the duplex. As presented in this disclosure, the direct repeat (DR) sequence originates from a short, repeated DNA sequence element within the CRISPR array. This sequence is typically interspersed between spacer sequences, which are derived from foreign genetic material such as phage or plasmid DNA. Upon transcription, the DR sequence is part of the pre-crRNA transcript and is processed into mature CRISPR RNA (crRNA). The DR sequence in the crRNA serves as a critical component that pairs with a complementary sequence in the trans-activating CRISPR RNA (tracrRNA) to form a double-stranded RNA (dsRNA) duplex. This duplex facilitates the binding of the Cas protein and is essential for the function of the guide RNA (gRNA) complex in the CRISPR/Cas system. In the context of the present disclosure, the DR sequence comprises the portion of the gRNA that hybridizes with the tracrRNA sequence to form the dsRNA duplex, thereby enabling the formation of the Cas protein binding segment. “hybridizes to form a double-stranded RNA (dsRNA) duplex”, or similar expressions, in this disclosure, refers to the process by which the direct repeat (DR) sequence of the CRISPR RNA (crRNA) pairs with a complementary sequence in the trans-activating CRISPR RNA (tracrRNA) to form a stable double-stranded RNA structure. The DR sequence in the crRNA and the complementary sequence in the tracrRNA undergo base pairing, forming hydrogen bonds between their complementary bases, resulting in the formation of the dsRNA duplex.

In some embodiments, the guide RNA is a dual guide RNA.

In some embodiments, the gRNA further comprises a linker sequence connecting the tracrRNA sequence and the DR sequence to form a sgRNA scaffold. In some typical embodiments, the linker comprises a short sequence of GAAA. In some embodiments, the linker serves as an artificial loop. In some embodiments, the sgRNA comprises, in an arrangement: (a) a spacer sequence, which is capable of hybridizing to a sequence of the target nucleic acid to be manipulated; (b) a DR sequence; (c) a linker sequence and (d) tracrRNA sequence. The tandem arrangement of the spacer sequence, the DR sequence, the linker sequence and tracrRNA sequence is in a 5′ to 3′ orientation, or in a 3′ to 5′orientation.

In some embodiments, the tracrRNA sequence is modified to lead the system having an enhancing activity when compared to that with wild type sequence (SEQ ID NO: 821). In some embodiments, the tracrRNA sequence is modified to enhance the stability of the gRNA when compared to that with wild type sequence (SEQ ID NO: 821). In some embodiments, the tracrRNA sequence is modified to reduce the interaction between the spacer sequence and the tracrRNA sequence, optionally, the modification of the tracrRNA sequence comprises the group consisting of: one or more nucleotides mutation, one or more nucleotides insertion, one or more nucleotides deletion. In some embodiments, the tracrRNA is modified to make the sequence comprise at least one stabilized hairpin secondary structure at a position that does not interfere with oligonucleotide binding and/or editing. In some embodiments, i) the stabilized hairpin forms a secondary structure comprising a contiguous stem having a length of 1, 2, 3, 4 or 5 nt more than that of the wild type sequence (SEQ ID NO: 821), and/or (ii) the stabilized hairpin forms a secondary structure comprising a contiguous stem having 1 C-G base pairs, at least 2 C-G base pairs, at least 3 C-G base pairs, at least 4 C-G base pairs, or at least 5 C-G base pairs more than that of the wild type sequence (SEQ ID NO: 821). In some embodiments, the tracrRNA sequence comprises a sequence having 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 821-826.

In some embodiments, the sgRNA scaffold comprises a sequence having 100%, or at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 841-846.

In some embodiments, the spacer sequence hybridizes to one or more nucleic acid in a prokaryotic cell or in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the eukaryotic cell comprises a mammalian cell. In some embodiments, the mammalian cell comprises a human cell. In some embodiments, the eukaryotic cell comprises a plant cell.

In some embodiments, the polynucleotide encoding the Cas protein or fusion protein is operably linked to a promoter; optionally, the promoter is a constitutive promoter, a tissue-specific promoter, or an inducible promoter. In some embodiments, the polynucleotide encoding the Cas protein or fusion protein is operably linked to a promoter and is present in a vector; optionally, the vector is selected from the group consisting of: a retroviral vector, a lentiviral vector, a phage vector, an adenoviral vector, an adeno-associated virus vector, a herpes simplex virus vector and a plasmid vector.

This disclosure also provides a gRNA comprising the features as defined above.

This disclosure also provides an engineered, non-naturally occurring polynucleotide encoding the Type II CRISPR-associated (Cas) protein or fusion protein as disclosed herein.

As presented in this disclosure, the terms “polynucleotide” refers to a polymeric form of nucleotides of any length, either deoxyribonucleotides or ribonucleotides, or analogs thereof. Polynucleotides may have any three-dimensional structure, and may perform any function, known or unknown. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. In some embodiments, the polynucleotide encoding more than one portion of an expressed the type II Cas protein herein can be operably linked to each other and relevant regulatory sequences (such as promoters, enhancers, and termination regions). For example, there can be a functional linkage between a regulatory sequence and an exogenous nucleic acid sequence resulting in expression of the latter. For another embodiment, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame. In some embodiments, the promoter is a constitutive promoter, a tissue-specific promoter, or an inducible promoter. In some embodiments, the term further can include all introns and other DNA sequences spliced from the mRNA transcript, along with variants resulting from alternative splice sites. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type.

In some embodiments, the polynucleotide encoding the Cas protein or fusion protein is operably linked to a promoter and is presented in a vector; optionally, the vector is selected from the group consisting of: a retroviral vector, a lentiviral vector, a phage vector, an adenoviral vector, an adeno-associated virus vector, a herpes simplex virus vector and a plasmid vector.

In some embodiments, the polynucleotide is a ribonucleotide sequence or a deoxyribonucleotide sequence, or analogs thereof; optionally, the polynucleotide is codon-optimized for expression in a cell of interest; In some embodiments, the polynucleotide is codon optimized for expression in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell, preferably a human cell. In some embodiments, the cell is a mammalian cell, preferably a human cell.

In some embodiments, the polynucleotide is an mRNA and further comprises a 5′ cap sequence and/or a poly-A tail sequence. In some embodiments of the present disclosure, the mRNA utilized may be modified to enhance its functional properties and stability. Specifically, in some embodiments, the modification process involves the substitution of uridine (represented by the letter “U”) with N1-Methylpseudouridine or pseudouridine. This substitution is designed to improve the mRNA's resistance to degradation by ribonucleases, potentially increasing its half-life and translational efficiency within the cell. The incorporation of N1-Methylpseudouridine or pseudouridine into the mRNA structure can also positively influence the immune response profile, as these modifications have been shown to reduce the immunogenicity of mRNA molecules when compared to their unmodified counterparts. This is particularly crucial for the development of mRNA-based therapeutics and vaccines, where minimizing adverse immune reactions is paramount.

In some embodiments, the polynucleotide in this disclosure is codon-optimized for expression in a eukaryotic cell; optionally, the eukaryotic cell is selected from the group consisting of: a plant cell, a fungal cell, a single-cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

As presented in this disclosure, the term “target nucleic acid” refers to a specific nucleic acid substrate that contains a nucleic acid sequence complement to the entirety or a part of the spacer in an RNA guide. In some embodiments, the target nucleic acid comprises a gene or a sequence within a gene. In certain embodiments, the target nucleic acid comprises a noncoding region (e.g., a promoter). In a specific embodiment, the target nucleic acid is single-stranded. In a specific embodiment, the target nucleic acid is double-stranded. The term “target nucleic acid” or “target sequence” needs to be understood according to the context in the disclosure.

In some embodiments, the guide RNA is a dual guide RNA. In some embodiments, the guide RNA is a single guide RNA. In such embodiments, the guide RNA further comprises a linker sequence connecting the tracrRNA sequence and the DR sequence. In some typical embodiments, the linker comprises a short sequence of GAAA. In some embodiments, the linker serves as an artificial loop. In some embodiments, the sgRNA comprises, in an arrangement: a) a spacer sequence, which is capable of hybridizing to a sequence of the target nucleic acid to be manipulated; b) a DR sequence; c) a linker sequence and d) tracrRNA sequence. The tandem arrangement of the spacer sequence, the DR sequence, the linker sequence and tracrRNA sequence is in a 5′ to 3′ orientation, or in a 3′ to 5′orientation; In some embodiments, the sgRNA scaffold comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NOs: 841-846.

In some embodiments, the spacer sequence hybridizes to one or more nucleic acid in a prokaryotic cell or in a eukaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the eukaryotic cell comprises a mammalian cell. In some embodiments, the mammalian cell comprises a human cell. In some embodiments, the eukaryotic cell comprises a plant cell.

In some embodiments, the system further comprising a donor template nucleic acid.

As presented in this disclosure, the term “donor template nucleic acid” as used herein refers to a nucleic acid molecule that can be used by one or more cellular proteins to alter the structure of a target nucleic acid after a Cas protein described herein has altered a target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome). In some embodiments, the donor template nucleic acid is a DNA or an RNA or a DNA-RNA hybrid.

This disclosure also provides an engineered vector comprising the polynucleotide described herein.

As presented in this disclosure, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. Common expression vectors of utility in recombinant DNA techniques are often in the form of plasmids. Recombinant expression vectors can comprise a nucleic acid of the disclosure in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). In some embodiments, the vector is an expression vector. In some embodiments, the vector is an inducible, conditional, or constitutive expression vector. In some embodiments, the polynucleotide encoding the Cas protein and the polynucleotides encoding the guide RNA are on a same vector or on different vectors.

This disclosure also provides a vector system comprising one or more polynucleotides described herein and one or more polynucleotides encoding a guide RNA; wherein said guide RNA comprises a spacer sequence that is complementary to a target nucleic acid and a Cas protein binding segment that interacts with said Cas protein, wherein the Cas protein binding segment comprises a tracrRNA sequence and a direct repeat (DR) sequence, and wherein the tracrRNA sequence hybridizes with the DR sequence to form a double-stranded RNA (dsRNA) duplex. In some embodiments, the polynucleotide encoding the Cas protein and the polynucleotides encoding the guide RNA are on a same vector or on different vectors.

This disclosure also provides an engineered, non-naturally occurring cell comprising: the Cas protein described in this disclosure, the polynucleotide described in this disclosure, the CRISPR-Cas system described in this disclosure, the vector described in this disclosure, or the vector system described in this disclosure.

This disclosure also provides a cell modified by utilizing the Cas protein described in this disclosure, the polynucleotide described in this disclosure, the CRISPR-Cas system described in this disclosure, the vector described in this disclosure, or the vector system described in this disclosure.

In some embodiments, the cell is an isolated eukaryotic cell wherein a target locus of interest is modified.

In some embodiments, the cell is a eukaryotic cell or a prokaryotic cell. In some embodiments, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some embodiments, the cell is a mammalian cell or a human cell or a plant cell.

In some embodiments, the cell is a vertebrate, mammalian, rodent, goat, pig, bird, chicken, turkey, cow, horse, sheep, fish, primate, or human cell. In some embodiments, the cell is a mammalian cell. In some embodiments, the cell is a human cell. In some embodiments, the cell is a somatic cell, a germ cell, or a prenatal cell. In some embodiments, the cell is a zygotic cell, a blastocyst cell, an embryonic cell, a stem cell, a mitotically competent cell, or a meiotically competent cell. In some embodiments, the cell is not part of a human embryo. In some embodiments, the cell is a somatic cell. In one embodiment, the cell is a T cell, a CD8+ T cell, a CD8+ naive T cell, a central memory T cell, an effector memory T cell, a CD4+ T cell, a stem cell memory T cell, a helper T cell, a regulatory T cell, a cytotoxic T cell, a natural killer T cell, a Hematopoietic Stem Cell, a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In some embodiments, the cell is a T cell, a Hematopoietic Stem Cell, a retinal cell, a cochlear hair cell, a pulmonary epithelial cell, a muscle cell, a neuron, a mesenchymal stem cell, an induced pluripotent stem (iPS) cell, or an embryonic stem cell. In another embodiment, the cell is a plant cell.

In some embodiments, said modifying of the target locus comprises inducing a DNA strand break. In some embodiments, said modifying a target locus comprises inducing a DNA double strand break or a DNA single strand break. In some embodiments, said modifying a target locus comprises altering gene expression of one or more genes. In some embodiments, said modifying a target locus comprises epigenetic modification of said target DNA locus.

In another aspect, this disclosure also provides a kit comprising: the Cas protein or fusion protein described herein, the polynucleotide described herein, the CRISPR-Cas system described herein, the vector described herein, the vector system described herein, or the cell described herein.

The kits described in this disclosure may encompass one or more containers with components essential for performing the methods in this disclosure, and may optionally contain instructions for use. Any of the kits delineated may additionally comprise ancillary components required for the execution of the editing methods. Each component within the kits, where applicable, may be provided in a liquid form (e.g., dissolved in solution) or in a solid form (e.g., lyophilized powder). In specific embodiments, some components may be reconstituted or otherwise processed (e.g., to an active state) upon the addition of a suitable solvent or other substance (such as water or buffer), which may or may not be furnished with the kit. In some embodiments, the kit may further comprise other suitable excipients such as buffers or reagents for facilitating the application of the kit. Preferably, the kit may be applied in various applications such as medical applications including therapies and diagnosis, researches and the like. Accordingly, the kit of the present disclosure may be used in the preparation of a medicament for treatment and/or in the preparation of an agent for research study.

The Cas protein described herein, fusion protein described herein, CRISPR-Cas system described herein, polynucleotide described herein, can be delivered by various delivery systems such as vectors, e.g., plasmids, viral delivery vectors, such as adeno-associated viruses (AAV), lentiviruses, adenoviruses, and other viral vectors, or methods, such as nucleofection or electroporation of ribonucleoprotein complexes consisting of Type V-I effectors and their cognate RNA guide or guides. The proteins and one or more RNA guides can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In some other aspects, this disclosure also provides a pharmaceutical composition comprising: (a) the Cas protein described herein; (b) the fusion protein described herein; (c) the polynucleotide described herein; (d) the CRISPR-Cas system described herein; (e) the vector described herein; (f) the vector system described herein; or (g) the cell described herein.

In some embodiments, the pharmaceutical composition further comprises a delivery system selected from: AAV (adeno-associated viruses), Adenoviruses, retroviruses, HSV (herpes simplex virus), Gammaretrovirus, LV (lentivirus), eCIS (extracellular Contractile Injection System), VLPs (virus-like particles), liposomes, plasmid, LNPs (lipid nanoparticles), exosomes, microvesicles, nucleic acid nanoassemblies, a gene gun, and an implantable device.

In some other aspects, this disclosure also provides the use of the Cas protein described herein; the fusion described herein the polynucleotide described herein, the CRISPR-Cas system described herein, the vector described herein, the vector system described herein, the cell described herein, the kit described herein, or the pharmaceutical composition described herein for the treatment, prevention, diagnosis, or detection of a disease.

In some other aspects, this disclosure also provides a method of modifying or targeting a target DNA locus, wherein the method comprising delivering to said locus: the Cas protein described herein; the fusion protein described herein; the polynucleotide described herein; the CRISPR-Cas system described herein; the vector described herein; the vector system described herein; the kit described herein or the pharmaceutical composition described herein.

In some embodiments, said modifying or targeting a target locus comprises inducing a DNA strand break. In some embodiments, said modifying or targeting a target locus comprises inducing a DNA double strand break or a DNA single strand break. In some embodiments, said modifying or targeting a target locus comprises altering gene expression of one or more genes. In some embodiments, said modifying or targeting a target locus comprises epigenetic modification of said target DNA locus. In some embodiments, the method is a method of modifying a cell, a cell line, or an organism by manipulation of one or more target sequences at genomic loci of interest.

In some other aspects, this disclosure also provides a method of cleaving a target DNA, the method comprising: contacting the target DNA with the Cas protein described herein; the polynucleotide described herein; the CRISPR-Cas system described herein; the vector described herein; the vector system described herein; the kit described herein or the pharmaceutical composition described herein.

In some embodiments, cleaving the target DNA sequence results in the formation of an indel or the insertion of a nucleotide sequence. In some embodiments, cleaving the target DNA or target nucleotide comprising cleaving the target DNA or target sequence in two sites, and results in the deletion or inversion of a sequence between the two sites. In some embodiments, the target DNA is a double stranded DNA or a single stranded DNA, or DNA-RNA hybrids.

In some embodiments, said modifying or targeting a target locus comprises inducing a DNA strand break, altering gene expression of one or more genes, or epigenetic modification of said target DNA locus; optionally, the DNA strand break comprise a DNA double strand break or a DNA single strand break.

In some embodiments, the method is performed ex vivo or in vivo.

This disclosure also provides an isolated eukaryotic cell comprising a modified target locus of interest, wherein the target locus of interest has been modified according to the method described in this disclosure.

This disclosure also provides a system for detecting the presence of a nucleic acid target sequence in an in vitro sample, comprising:
(a) a Cas protein described in this disclosure;
(b) at least one guide polynucleotide comprising a spacer sequence capable of binding the target sequence, and designed to form a complex with the Cas protein; and
(c) a nucleic acid-based masking construct comprising a non-target sequence; wherein the Cas protein exhibits collateral cleavage activity of RNA and/or ssDNA and cleaves the non-target sequence of the nucleic acid-based masking construct activated by the target sequence.

This disclosure also provides a method for detecting target nucleic acids in samples comprising: (a) contacting one or more samples with
(i) a Cas protein described in this disclosure;
(ii) at least one guide polynucleotide comprising a guide sequence designed to have a degree of complementarity with the target sequence, and designed to form a complex with the Cas protein; and
(iii) a nucleic acid-based masking construct comprising a non-target sequence; wherein the Cas protein exhibits collateral cleavage activity of RNA and/or ssDNA and cleaves the non-target sequence of the nucleic acid-based masking construct activated by the target sequences; and
(b) detecting a signal from cleavage of the non-target sequence, thereby detecting the one or more target sequences in the sample.

The wild-type Cas proteins identified are shown in Table 1; and the corresponding DNA sequences encoding them designated SEQ ID NOs: 101-177. The tracr sequences, the DR sequences and the sgRNA scaffold sequences for the corresponding Cas proteins are shown in Tables 3-5.

The specific Cas protein GEBx0530 and its mutation variants were shown in Table 2; and the corresponding DNA sequences encoding them designated SEQ ID NOs: 181-195.

TABLE 1
The amino acid sequences of the exemplary Cas proteins

| Name | SEQ ID NO |
|---|---|
| GEBx0462 | 1 |
| GEBx0463 | 2 |
| GEBx0464 | 3 |
| GEBx0465 | 4 |
| GEBx0466 | 5 |
| GEBx0467 | 6 |
| GEBx0468 | 7 |
| GEBx0469 | 8 |
| GEBx0470 | 9 |
| GEBx0471 | 10 |
| GEBx0472 | 11 |
| GEBx0473 | 12 |
| GEBx0474 | 13 |
| GEBx0475 | 14 |
| GEBx0476 | 15 |
| GEBx0477 | 16 |
| GEBx0478 | 17 |
| GEBx0479 | 18 |
| GEBx0480 | 19 |
| GEBx0481 | 20 |
| GEBx0482 | 21 |
| GEBx0483 | 22 |
| GEBx0484 | 23 |
| GEBx0485 | 24 |
| GEBx0486 | 25 |
| GEBx0487 | 26 |
| GEBx0488 | 27 |
| GEBx0489 | 28 |
| GEBx0490 | 28 |
| GEBx0491 | 30 |
| GEBx0492 | 31 |
| GEBx0493 | 32 |
| GEBx0494 | 33 |
| GEBx0495 | 34 |
| GEBx0496 | 35 |
| GEBx0497 | 36 |
| GEBx0498 | 37 |
| GEBx0499 | 38 |
| GEBx0500 | 39 |
| GEBx0501 | 40 |
| GEBx0502 | 41 |
| GEBx0503 | 42 |
| GEBx0504 | 43 |
| GEBx0505 | 44 |
| GEBx0506 | 45 |
| GEBx0507 | 46 |
| GEBx0508 | 47 |
| GEBx0509 | 48 |
| GEBx0510 | 49 |
| GEBx0511 | 50 |
| GEBx0512 | 51 |
| GEBx0513 | 52 |
| GEBx0514 | 53 |
| GEBx0515 | 54 |
| GEBx0516 | 55 |
| GEBx0517 | 56 |
| GEBx0518 | 57 |
| GEBx0519 | 58 |
| GEBx0520 | 59 |
| GEBx0521 | 60 |
| GEBx0522 | 61 |
| GEBx0523 | 62 |
| GEBx0524 | 63 |
| GEBx0525 | 64 |
| GEBx0526 | 65 |
| GEBx0527 | 66 |
| GEBx0528 | 67 |
| GEBx0529 | 68 |
| GEBx0530 | 69 |
| GEBx0531 | 70 |
| GEBx0532 | 71 |
| GEBx0533 | 72 |
| GEBx0534 | 73 |
| GEBx0535 | 74 |
| GEBx0536 | 75 |
| GEBx0537 | 76 |
| GEBx0538 | 77 |

TABLE 2
The amino acid sequences of GEBx0530 and its mutation variants.

| Name | SEQ ID NO | Sequences |
|---|---|---|
| GEBx0530- | 69 | MSIAEKNNSEYYLGLDIGTDSVGWAVTDLDYNIQKFNGKA |
| wt | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLKLSNN |
| | | FSNRKSFILYNQSITGLFEQKVDLLRL |
| GEBx0530- | 81 | MSIAEKNNSEYYLGLDIGTDSVGWAVTDLDYNIQKFNGKA |
| K1315Q | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLQLSNN |
| | | FSNRKSFILYNQSITGLFEQKVDLLRL |
| GEBx0530- | 82 | MSIAEKNNSEYYLGLDIGTDSVGWAVTDLDYNIQKFNGKA |
| K1315N | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLNLSNN |
| | | FSNRKSFILYNQSITGLFEQKVDLLRL |
| GEBx0530- | 83 | MSIAEKNNSEYYLGLDIGTDSVGWAVTDLDYNIQKFNGKA |
| K1315T | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLTLSNN |
| | | FSNRKSFILYNQSITGLFEQKVDLLRL |
| GEBx0530- | 84 | MSIAEKNNSEYYLGLDIGTDSVGWAVTDLDYNIQKFNGKA |
| A1113N | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KANGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLKLSNN |
| | | FSNRKSFILYNQSITGLFEQKVDLLRL |
| GEBx0530- | 85 | MSIAEKNNSEYYLGLDIGTDSVGWAVTDLDYNIQKFNGKA |
| A1113K | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAKGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLKLSNN |
| | | FSNRKSFILYNQSITGLFEQKVDLLRL |
| GEBx0530- | 86 | MSIAEKNNSEYYLGLDIGTDSVGWAVTDLDYNIQKFNGKA |
| A1113R | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KARGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLKLSNN |
| | | FSNRKSFILYNQSITGLFEQKVDLLRL |
| GEBx0530- | 87 | MSIAEKNNSEYYLGLDIGTDSVGWAVTDLDYNIQKENGKA |
| Q1191N | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKNVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLKLSNN |
| | | FSNRKSFILYNQSITGLFEQKVDLLRL |
| GEBx0530- | 88 | MSIAEKNNSEYYLGLDIGTDSVGWAVTDLDYNIQKFNGKA |
| Q1191K | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKKVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLKLSNN |
| | | FSNRKSFILYNQSITGLFEQKVDLLRL |
| GEBx0530- | 89 | MSIAEKNNSEYYLGLDIGTDSVGWAVTDLDYNIQKFNGKA |
| Q1191V | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKVVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLKLSNN |
| | | FSNRKSFILYNQSITGLFEQKVDLLRL |
| GEBx0530- | 90 | MSIAEKNNSEYYLGLDIGTDSVGWAVTDLDYNIQKFNGKA |
| L1298R | | MWGIRLFEAGKTAAERRVFRTTRRRIQRRKHRIELLQELFDS |
| | | EISKADTNFYLRLKESKYWVEDRTLGQTNILFNDPEYTDKD |
| | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSRSANFKLINSGASCGTLKLSNN |
| | | FSNRKSFILYNQSITGLFEQKVDLLRL |
| GEBx0530- | 91 | MSIAEKNNSEYYLGLDIGTDSVGWAVTDLDYNIQKFNGKA |
| DM3 | | MWGIRLFEAGKTAAERRVFRTTRRRIQRRKHRIELLQELFDS |
| (A1113R/ | | EISKADTNFYLRLKESKYWVEDRTLGQTNILFNDPEYTDKD |
| K1315Q) | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KARGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLQLSNN |
| | | FSNRKSFILYNQSITGLFEQKVDLLRL |
| GEBx0530- | 92 | MSIAEKNNSEYYLGLDIGTDSVGWAVTDLDYNIQKENGKA |
| DM6 | | MWGIRLFEAGKTAAERRVFRTTRRRIQRRKHRIELLQELFDS |
| (L1298R/ | | EISKADTNFYLRLKESKYWVEDRTLGQTNILFNDPEYTDKD |
| K1315Q) | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KAAGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSRSANFKLINSGASCGTLQLSN |
| | | NFSNRKSFILYNQSITGLFEQKVDLLRL |
| GEBx0530- | 93 | MSIAEKNNSEYYLGLDIGTDSVGWAVTDLDYNIQKFNGKA |
| DM7 | | MWGIRLFEAGKTAAERRVFRTTRRRIQRRKHRIELLQELFDS |
| (A1113R/ | | EISKADTNFYLRLKESKYWVEDRTLGQTNILFNDPEYTDKD |
| K1315T) | | YYKQYPTIYHLRKELMTSDEPHDVRLVYLAIHHLIKNRGHF |
| | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KARGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSLSANFKLINSGASCGTLTLSNN |
| | | FSNRKSFILYNQSITGLFEQKVDLLRL |
| GEBx0530- | 94 | MSIAEKNNSEYYLGLDIGTDSVGWAVTDLDYNIQKFNGKA |
| TM1 | | MWGIRLFEAGKTAAERRVFRTTRRRIQRRKHRIELLQELFDS |
| (A1113R/ | | EISKADTNFYLRLKESKYWVEDRTLGQTNILFNDPEYTDKD |
| L1298R/ | | YYKQYPTIYHLRKELMTSDEPHDVRLVYLAIHHLIKNRGHF |
| K1315Q) | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KARGAYFFLVEHTVKKKRIRTLEFAPVYLAKKLENRSALLEY |
| | | CKNELNLLESRILLPKVKINTMFEIDGFKMHISGRTGKQVIFK |
| | | GAEQLCISEQYGLDIKKINKYIQRCKLARENLPVTAFDGISTH |
| | | NNDVLYSVMMQKLKDTVYGIHLSSQLKNLEKGKEVFGKLS |
| | | LKEQCEVLMEALNLFSCNSRSANFKLINSGASCGTLQLSNNF |
| | | SNRKSFILYNQSITGLFEQKVDLLRL |
| GEBx0530- | 95 | MSIAEKNNSEYYLGLDIGTDSVGWAVTDLDYNIQKFNGKA |
| TM2 | | MWGIRLFEAGKTAAERRVFRTTRRRIQRRKHRIELLQELFDS |
| (A1113R/ | | EISKADTNFYLRLKESKYWVEDRTLGQTNILFNDPEYTDKD |
| L1298R/ | | YYKQYPTIYHLRKELMTSDEPHDVRLVYLAIHHLIKNRGHF |
| K1315T) | | LFEGEGRKDVSSFKVIFRELCQSMHDEMEIVLEAADLDEVE |
| | | AVLKNREYGITDKKTRLNKLLTIDDEHKKEQKSIIALLAGGK |
| | | TKVDALFGEKTSEDSDVKDITFAGDKFEESFDALSIMLGDK |
| | | MFCLEKMKAIYDWSILEEILQGEDYLSYAKVATYEKHKNDL |
| | | ATLKKIMKKYRSDEYDACFKDPGKKDNYCAYVGMAKKGK |
| | | KKLVLEKKCSQEDLCKYLGKVFKDVKQNDPDLQYLMAEIQ |
| | | RGTLLPKQVNKDNGVIPHQLHLEELRMILANVKKYLEFLND |
| | | KDDSGFTVSEKIEKIFAFRIPYYVGPLNAHHKSLGHCWIVRN |
| | | KGMENQKVLPWNFDEIVNLEASATAFIRKMTSKCTYVIGAD |
| | | VVPKNSLVYSEFMVLNELNNVKVNGELLPVELKEKVFEEVF |
| | | KRVKRVTGKRLRDFLIAEGAFKKGDELSGFDQNFKGSLTAY |
| | | LDFKRILGEKKIPKEAIEEMIQAIVLFGDDRKLLKRRVEKRYG |
| | | ELLSKEEIAAICKLKYTGWGRLSREFLEEIYVVNHKTGEMIN |
| | | IISMMHGTNANLMQLLSSEYRFTEALEAYNEERQTDHSDIAY |
| | | GIVKDLYVSPAVRRSIWQSLKIVKEIAKIKKAAPKKVFVEVA |
| | | RENAEKKRTVSRKNALIALYKNCKEEERDWIAELDAKTDHE |
| | | LRRDRLYLYYTQMGRCMYTGKPIDLNRLFDTNVYDVDHIFP |
| | | QSKIKDDSLSNRVLVERKVNAKKSDVYPLPEEIRNKNRGFW |
| | | TMLHRKELISKRKYDRLTRVTPFSDSELADFIARQLVETRQST |
| | | KAVAQVLKKVFPDTDVVYVKAGNVSRFRYDFKMIKVREVN |
| | | DYHHAKDAYLNIVVGNVYDTKFTKSPLNFIQGKDGKNYSL |
| | | NQMFNYDVERSGVIAWKAGEKGTITTVKRMMKKNNIQFTR |
| | | HAFEVKGGLFDQTIMKKGKGQVPIKSSDVHLSDISKYGGYN |
| | | KARGAYFFLVEHTVKKKRIRTLEFAPVYLAKKLENRSALLE |
| | | YCKNELNLLESRILLPKVKINTMFEIDGFKMHISGRTGKQVIF |
| | | KGAEQLCISEQYGLDIKKINKYIQRCKLARENLPVTAFDGIST |
| | | HNNDVLYSVMMQKLKDTVYGIHLSSQLKNLEKGKEVFGKL |
| | | SLKEQCEVLMEALNLFSCNSRSANFKLINSGASCGTLTLSNN |
| | | FSNRKSFILYNQSITGLFEQKVDLLRL |

TABLE 3
The tracrRNA sequence of the corresponding Cas proteins:

| Name | SEQ ID NO: |
|---|---|
| GEBx0462/HPT-V1 | 201 |
| GEBx0463/HPT-V1 | 202 |
| GEBx0464/HPT-V1 | 203 |
| GEBx0465/HPT-V1 | 204 |
| GEBx0466/HPT-V1 | 205 |
| GEBx0467/HPT-V1 | 206 |
| GEBx0468/HPT-V1 | 207 |
| GEBx0468/HPT-V2 | 208 |
| GEBx0469/HPT-V1 | 209 |
| GEBx0470/HPT-V1 | 210 |
| GEBx0471/HPT-V1 | 211 |
| GEBx0472/HPT-V1 | 212 |
| GEBx0473/HPT-V1 | 213 |
| GEBx0474/HPT-V1 | 214 |
| GEBx0475/HPT-V1 | 215 |
| GEBx0476/HPT-V1 | 216 |
| GEBx0477/HPT-V1 | 217 |
| GEBx0478/HPT-V1 | 218 |
| GEBx0479/HPT-V1 | 219 |
| GEBx0480/HPT-V1 | 220 |
| GEBx0481/HPT-V1 | 221 |
| GEBx0482/HPT-V1 | 222 |
| GEBx0483/HPT-V1 | 223 |
| GEBx0484/HPT-V1 | 224 |
| GEBx0485/HPT-V1 | 225 |
| GEBx0486/HPT-V1 | 226 |
| GEBx0487/HPT-V1 | 227 |
| GEBx0488/HPT-V1 | 228 |
| GEBx0489/HPT-V1 | 229 |
| GEBx0489/HPT-V2 | 230 |
| GEBx0490/HPT-V1 | 231 |
| GEBx0491/HPT-V1 | 232 |
| GEBx0491/HPT-V2 | 233 |
| GEBx0492/HPT-V1 | 234 |
| GEBx0493/HPT-V1 | 235 |
| GEBx0494/HPT-V1 | 236 |
| GEBx0495/HPT-V1 | 237 |
| GEBx0496/HPT-V1 | 238 |
| GEBx0497/HPT-V1 | 239 |
| GEBx0498/HPT-V1 | 240 |
| GEBx0499/HPT-V1 | 241 |
| GEBx0500/HPT-V1 | 242 |
| GEBx0501/HPT-V1 | 243 |
| GEBx0502/HPT-V1 | 244 |
| GEBx0502/HPT-V2 | 245 |
| GEBx0503/HPT-V1 | 246 |
| GEBx0504/HPT-V1 | 247 |
| GEBx0505/HPT-V1 | 248 |
| GEBx0506/HPT-V1 | 249 |
| GEBx0507/HPT-V1 | 250 |
| GEBx0508/HPT-V1 | 251 |
| GEBx0509/HPT-V1 | 252 |
| GEBx0510/HPT-V1 | 253 |
| GEBx0511/HPT-V1 | 254 |
| GEBx0512/HPT-V1 | 255 |
| GEBx0513/HPT-V1 | 256 |
| GEBx0514/HPT-V1 | 257 |
| GEBx0515/HPT-V1 | 258 |
| GEBx0516/HPT-V1 | 259 |
| GEBx0517/HPT-V1 | 260 |
| GEBx0517/HPT-V2 | 261 |
| GEBx0518/HPT-V1 | 262 |
| GEBx0519/HPT-V1 | 263 |
| GEBx0520/HPT-V1 | 264 |
| GEBx0520/HPT-V2 | 265 |
| GEBx0521/HPT-V1 | 266 |
| GEBx0522/HPT-V1 | 267 |
| GEBx0523/HPT-V1 | 268 |
| GEBx0524/HPT-V1 | 269 |
| GEBx0525/HPT-V1 | 270 |
| GEBx0526/HPT-V1 | 271 |
| GEBx0527/HPT-V1 | 272 |
| GEBx0528/HPT-V1 | 273 |
| GEBx0529/HPT-V1 | 274 |
| GEBx0530/HPT-V1 | 275 |
| GEBx0531/HPT-V1 | 276 |
| GEBx0532/HPT-V1 | 277 |
| GEBx0533/HPT-V1 | 278 |
| GEBx0534/HPT-V1 | 279 |
| GEBx0535/HPT-V1 | 280 |
| GEBx0536/HPT-V1 | 281 |
| GEBx0536/HPT-V2 | 282 |
| GEBx0538/HPT-V1 | 283 |

The nucleic acids of the direct repeat sequence (DR) are shown in Table 4.

TABLE 4
The nucleotide sequence of the DR described in the disclosure

| Name | SEQ ID NO: |
|---|---|
| GEBx0462/HPT-V1 | 301 |
| GEBx0463/HPT-V1 | 302 |
| GEBx0464/HPT-V1 | 303 |
| GEBx0465/HPT-V1 | 304 |
| GEBx0466/HPT-V1 | 305 |
| GEBx0467/HPT-V1 | 306 |
| GEBx0468/HPT-V1 | 307 |
| GEBx0468/HPT-V2 | 308 |
| GEBx0469/HPT-V1 | 309 |
| GEBx0470/HPT-V1 | 310 |
| GEBx0471/HPT-V1 | 311 |
| GEBx0472/HPT-V1 | 312 |
| GEBx0473/HPT-V1 | 313 |
| GEBx0474/HPT-V1 | 314 |
| GEBx0475/HPT-V1 | 315 |
| GEBx0476/HPT-V1 | 316 |
| GEBx0477/HPT-V1 | 317 |
| GEBx0478/HPT-V1 | 318 |
| GEBx0479/HPT-V1 | 319 |
| GEBx0480/HPT-V1 | 320 |
| GEBx0481/HPT-V1 | 321 |
| GEBx0482/HPT-V1 | 322 |
| GEBx0483/HPT-V1 | 323 |
| GEBx0484/HPT-V1 | 324 |
| GEBx0485/HPT-V1 | 325 |
| GEBx0486/HPT-V1 | 326 |
| GEBx0487/HPT-V1 | 327 |
| GEBx0488/HPT-V1 | 328 |
| GEBx0489/HPT-V1 | 329 |
| GEBx0489/HPT-V2 | 330 |
| GEBx0490/HPT-V1 | 331 |
| GEBx0491/HPT-V1 | 332 |
| GEBx0491/HPT-V2 | 333 |
| GEBx0492/HPT-V1 | 334 |
| GEBx0493/HPT-V1 | 335 |
| GEBx0494/HPT-V1 | 336 |
| GEBx0495/HPT-V1 | 337 |
| GEBx0496/HPT-V1 | 338 |
| GEBx0497/HPT-V1 | 339 |
| GEBx0498/HPT-V1 | 340 |
| GEBx0499/HPT-V1 | 341 |
| GEBx0500/HPT-V1 | 342 |
| GEBx0501/HPT-V1 | 343 |
| GEBx0502/HPT-V1 | 344 |
| GEBx0502/HPT-V2 | 345 |
| GEBx0503/HPT-V1 | 346 |
| GEBx0504/HPT-V1 | 347 |
| GEBx0505/HPT-V1 | 348 |
| GEBx0506/HPT-V1 | 349 |
| GEBx0507/HPT-V1 | 350 |
| GEBx0508/HPT-V1 | 351 |
| GEBx0509/HPT-V1 | 352 |
| GEBx0510/HPT-V1 | 353 |
| GEBx0511/HPT-V1 | 354 |
| GEBx0512/HPT-V1 | 355 |
| GEBx0513/HPT-V1 | 356 |
| GEBx0514/HPT-V1 | 357 |
| GEBx0515/HPT-V1 | 358 |
| GEBx0516/HPT-V1 | 359 |
| GEBx0517/HPT-V1 | 360 |
| GEBx0517/HPT-V2 | 361 |
| GEBx0518/HPT-V1 | 362 |
| GEBx0519/HPT-V1 | 363 |
| GEBx0520/HPT-V1 | 364 |
| GEBx0520/HPT-V2 | 365 |
| GEBx0521/HPT-V1 | 366 |
| GEBx0522/HPT-V1 | 367 |
| GEBx0523/HPT-V1 | 368 |
| GEBx0524/HPT-V1 | 369 |
| GEBx0525/HPT-V1 | 370 |
| GEBx0526/HPT-V1 | 371 |
| GEBx0527/HPT-V1 | 372 |
| GEBx0528/HPT-V1 | 373 |
| GEBx0529/HPT-V1 | 374 |
| GEBx0530/HPT-V1 | 375 |
| GEBx0531/HPT-V1 | 376 |
| GEBx0532/HPT-V1 | 377 |
| GEBx0533/HPT-V1 | 378 |
| GEBx0534/HPT-V1 | 379 |
| GEBx0535/HPT-V1 | 380 |
| GEBx0536/HPT-V1 | 381 |
| GEBx0536/HPT-V2 | 382 |
| GEBx0538/HPT-V1 | 383 |

Direct Repeats (DR) and CRISPR RNA (crRNA) are indispensable components of the CRISPR/Cas system, a prokaryotic immune mechanism that defends against foreign genetic invaders. The transcription of the CRISPR array, which is composed of alternating DR sequences and unique spacer sequences, produces the pre-crRNA. This precursor is then processed into individual crRNAs, where each mature crRNA contains a spacer sequence flanked by DR segments. These crRNAs subsequently guide Cas proteins to recognize and cleave complementary target DNA sequences, demonstrating the crucial role of DR sequences in both the transcription and functional maturation of crRNAs. A portion of the crRNA sequence is transcribed from the DR disclosed herein. When the sequence disclosed herein refers to a crRNA, “T” should be understood as “U”. For example, in some embodiments, the disclosure provides an engineered, non-naturally occurring crRNA, wherein the crRNA comprises a nucleotide sequence having at least 90% sequence identity to any one of SEQ ID NOs: 801-883, or a variant thereof. In some embodiments, the crRNA comprises a nucleotide sequence having at least 95% or 98% sequence identity to any one of SEQ ID NOs: 801-883. In some embodiments, the crRNA comprises a nucleotide sequence set forth in any one of SEQ ID NOs: 801-883.

TABLE 5
The sequence encoding the sgRNA scaffold of the Cas proteins:

| Name | SEQ ID NO: |
|---|---|
| GEBx0462-HPT-V1 | 401 |
| GEBx0463-HPT-V1 | 402 |
| GEBx0464-HPT-V1 | 403 |
| GEBx0465-HPT-V1 | 404 |
| GEBx0466-HPT-V1 | 405 |
| GEBx0467-HPT-V1 | 406 |
| GEBx0468-HPT-V1 | 407 |
| GEBx0468-HPT-V2 | 408 |
| GEBx0469-HPT-V1 | 409 |
| GEBx0470-HPT-V1 | 410 |
| GEBx0471-HPT-V1 | 411 |
| GEBx0472-HPT-V1 | 412 |
| GEBx0473-HPT-V1 | 413 |
| GEBx0474-HPT-V1 | 414 |
| GEBx0475-HPT-V1 | 415 |
| GEBx0476-HPT-V1 | 416 |
| GEBx0477-HPT-V1 | 417 |
| GEBx0478-HPT-V1 | 418 |
| GEBx0479-HPT-V1 | 419 |
| GEBx0480-HPT-V1 | 420 |
| GEBx0481-HPT-V1 | 421 |
| GEBx0482-HPT-V1 | 422 |
| GEBx0483-HPT-V1 | 423 |
| GEBx0484-HPT-V1 | 424 |
| GEBx0485-HPT-V1 | 425 |
| GEBx0486-HPT-V1 | 426 |
| GEBx0487-HPT-V1 | 427 |
| GEBx0488-HPT-V1 | 428 |
| GEBx0489-HPT-V1 | 429 |
| GEBx0489-HPT-V2 | 430 |
| GEBx0490-HPT-V1 | 431 |
| GEBx0491-HPT-V1 | 432 |
| GEBx0491-HPT-V2 | 433 |
| GEBx0492-HPT-V1 | 434 |
| GEBx0493-HPT-V1 | 435 |
| GEBx0494-HPT-V1 | 436 |
| GEBx0495-HPT-V1 | 437 |
| GEBx0496-HPT-V1 | 438 |
| GEBx0497-HPT-V1 | 439 |
| GEBx0498-HPT-V1 | 440 |
| GEBx0499-HPT-V1 | 441 |
| GEBx0500-HPT-V1 | 442 |
| GEBx0501-HPT-V1 | 443 |
| GEBx0502-HPT-V1 | 444 |
| GEBx0502-HPT-V2 | 445 |
| GEBx0503-HPT-V1 | 446 |
| GEBx0504-HPT-V1 | 447 |
| GEBx0505-HPT-V1 | 448 |
| GEBx0506-HPT-V1 | 449 |
| GEBx0507-HPT-V1 | 450 |
| GEBx0508-HPT-V1 | 451 |
| GEBx0509-HPT-V1 | 452 |
| GEBx0510-HPT-V1 | 453 |
| GEBx0511-HPT-V1 | 454 |
| GEBx0512-HPT-V1 | 455 |
| GEBx0513-HPT-V1 | 456 |
| GEBx0514-HPT-V1 | 457 |
| GEBx0515-HPT-V1 | 458 |
| GEBx0516-HPT-V1 | 459 |
| GEBx0517-HPT-V1 | 460 |
| GEBx0517-HPT-V2 | 461 |
| GEBx0518-HPT-V1 | 462 |
| GEBx0519-HPT-V1 | 463 |
| GEBx0520-HPT-V1 | 464 |
| GEBx0520-HPT-V2 | 465 |
| GEBx0521-HPT-V1 | 466 |
| GEBx0522-HPT-V1 | 467 |
| GEBx0523-HPT-V1 | 468 |
| GEBx0524-HPT-V1 | 469 |
| GEBx0525-HPT-V1 | 470 |
| GEBx0526-HPT-V1 | 471 |
| GEBx0527-HPT-V1 | 472 |
| GEBx0528-HPT-V1 | 473 |
| GEBx0529-HPT-V1 | 474 |
| GEBx0530-HPT-V1 | 475 |
| GEBx0531-HPT-V1 | 476 |
| GEBx0532-HPT-V1 | 477 |
| GEBx0533-HPT-V1 | 478 |
| GEBx0534-HPT-V1 | 479 |
| GEBx0535-HPT-V1 | 480 |
| GEBx0536-HPT-V1 | 481 |
| GEBx0536-HPT-V2 | 482 |
| GEBx0538-HPT-V1 | 483 |

In some embodiments, the spacer sequence is between 10 and 40 nucleotides in length, preferably the spacer sequence is between 15 and 30 nucleotides in length, or between 18 and 25 nucleotides in length. In some embodiments, the spacer sequence is 15, 16, 17, 18, 19, 20, 21, 22, 23, 24, 25, 26, 27, 28, 29, or 30 nucleotides in length.

Although the description referred to particular embodiments and aspects, the disclosure should not be construed as limited to the embodiments set forth herein.

### EXAMPLES

### Example 1: A Method of Metagenomic Analysis for the Proteins

Metagenomic sequence data from public databases were search using Hidden Markov Models generated based on known Cas protein sequences including class 2 type II Cas proteins. CRISPR-Cas protein identified by the search are aligned to known proteins to identify potential active sites. From hundreds of potential sequences, finally, this metagenomic workflow results in the delineation of the Cas proteins listed in SEQ ID NOs: 1-77, Table 1. A phylogenetic tree was constructed by MUSCLE 3 (Veen et. al, 2020) to visualize the relatedness of the orthologs at the primary amino-acid level using 213 Class 2 Type II-A/B/C sequences from The National Center for Biotechnology Information (NCBI), various publications, and patents.

### Example 2: Protocol for Protein and sgRNA Folding

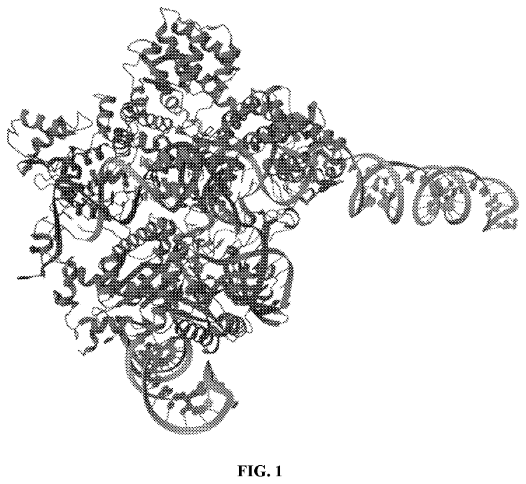

Prediction of protein and sgRNA complex folding is conducted utilizing AlphaFold 3 (Josh et. al. 2024. Nature). All Cas proteins disclosed herein encompass RuvC, BH (bridge helix), REC, HNH and CTD (C-terminal domain) domains. The RuvC domain contains three split RuvC sub-domains. FIG. 1 shows the RNP structure of GEBx0530 as an Example.

### Example 3: PAM Determination in Mammalian Cell Line

In a set of experiments, HEK293T cells were cultured in DMEM media supplemented with 10% fetal bovine serum (Gibco™). For reverse transfection, HEK293T cells were cultured in DMEM media supplemented with 10% fetal bovine serum (Gibco™). A volume of 450 μL of cells with a density of 100,000 cells/well was mixed with 50 μL mixture containing Lipofectamine™ 3000 (ThermoFisher Scientific, Cat. L3000008), Opti-Mem (Volume refill to 50 μL), 1 μL dsODN (10 pM), 100 ng (~1 μL) psgRNA harbored sgRNA scaffold and Humanspacer3 spacer (SEQ ID NO: 500) encoding sequence and 400 ng (~1 μL) pCasX plasmid harbored CDS encoding Cas proteins with NLS and FLAG (SEQ ID NOs: 521-597, Table 7) per the manufacturer's protocol. Then seeded the cell mixture onto a 24-well plate and cultured at 37° C. and 5% CO₂. 10 pM dsODN was annealed using dsODN-Top and dsODN-BOT oligonucleotides pre-transfection.

72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples (500 ng, 260/280 value: 1.8~2.0) were subjected to Guide-Seq NGS analyses.

The basic method of Guide-Seq library preparation is described by Nikolay et. al (Nat. Protoc. 2021). The extracted DNA sample were first sheared using KAPA Frag Kit (Cat #KK8602, Roche). Fragmented DNA was purified and then phosphorated using T4 Polynucleotide Kinase (Cat #M0201S, NEB). An SS5-adapter (generated by annealing 10 μM SS5TOP oligo with 10 μM SS5B™ oligo) was ligated to the fragmented DNA using Quick Ligation™ Kit (Cat #M2200S, NEB), followed by two steps off-target PCR to add chemistry for sequencing.

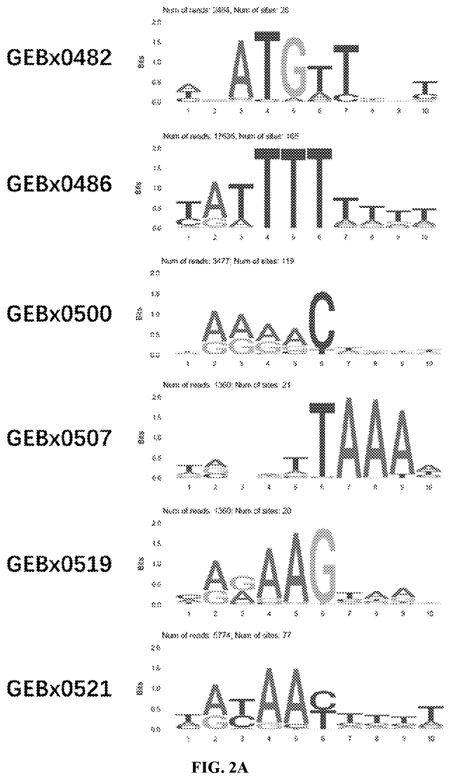

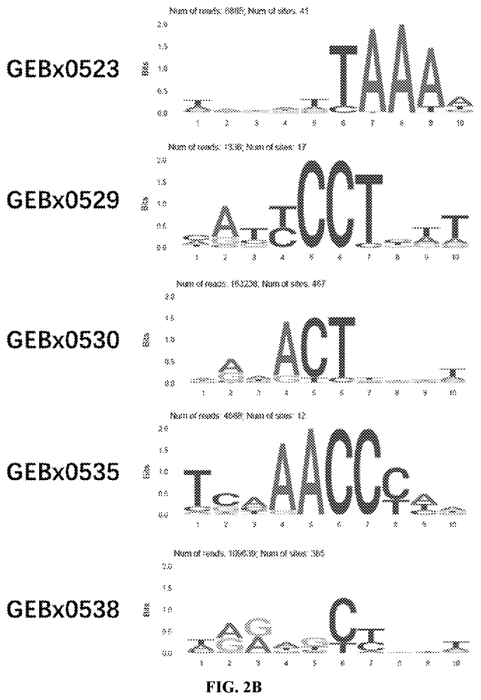

PCR1 was performed using Platinum™ Taq DNA Polymerase (Cat #15966005, Invitrogen) with GSP1 (a mixture of GSP1-Top and GSP1-BoT) and Y_XX oligos. PCR2 was performed using Platinum™ Taq DNA Polymerase with GSP2 (a mixture of GSP2-TopA/B/C and GSP1-BoTA/B/C), Y_XX (Same as PCR1) and i753_XX oligos. The DNA product in each step described need purification using SPRI Select (Cat #B23318, Beckman Coulter). The final library was quantified with qPCR and sequenced on Illumina NextSeq 1000. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected. The PAM preference of some exemplary Cas effector proteins in HEK293 cell line is shown in FIG. 2A an FIG. 2B. The nucleotide sequences used for PAM determination are listed in Table 6.

TABLE 6
The nucleotide sequences used for PAM determination

| | SEQ | |
|---|---|---|
| Name | ID NO: | 5′-3′ |
| Humanspacer3 | 500 | GTGAGCCACTGTGCCTGGCC |
| dsODN-Top | 501 | pACTTCGGTACGCGTCGACGTACGGTCGACGAGTCGCGCGTACGACGATCG |
| | | A*G*A*C |
| dsODN-BoT | 502 | pGTCTCGATCGTCGTACGCGCGACTCGTCGACCGTACGTCGACGCGTACCG |
| | | A*A*G*T |
| SSSTOP | 503 | A*A*C*TACACGACGCTCTTCCGATCTNNNWNNNWNNAGTGACAC |
| SS5BTM | 504 | NNNNNNGTGTCACTNNWNNNWNNNAGATCGGAAGAGCGTCGTGTAGTTp |
| GSP1-Top | 505 | GTCGACGAGTCGCGCGTA |
| GSP1-BoT | 506 | TCGTCGACCGTACGTCGA |
| GSP2-Top | 507 | GTGACTGGAGTTCAGACGTGTGCTCTTCCGATCTCGCGCGTACGACGAT*C* |
| A | | G*A |
| GSP2-Top | 508 | GTGACTGGAGTTCAGACGTGTGCTCTTCCGATCTATCGCGCGTACGACGAT* |
| B | | C*G*A |
| GSP2-Top | 509 | GTGACTGGAGTTCAGACGTGTGCTCTTCCGATCTTAACGCGCGTACGACGA |
| C | | T*C*G*A |
| GSP2-BoT | 510 | GTGACTGGAGTTCAGACGTGTGCTCTTCCGATCTTACGTCGACGCGTAC*C* |
| A | | G*A |
| GSP2-BoT | 511 | GTGACTGGAGTTCAGACGTGTGCTCTTCCGATCTGCTACGTCGACGCGTAC |
| B | | *C*G*A |
| GSP2-BoT | 512 | GTGACTGGAGTTCAGACGTGTGCTCTTCCGATCTATGTACGTCGACGCGTA |
| C | | C*C*G*A |
| Y_XX | 513 | AATGATACGGCGACCACCGAGATCTACACNNNNNNNNACACTCTTTCCCTA |
| | | CACGACGCTCTTCCGATCT |
| i753_XX | 514 | CAAGCAGAAGACGGCATACGAGATNNNNNNNNGTGACTGGAGTTCAGAC |
| | | GTGTGCTCTTCCGATCT |

Note:
p: phosphorylation modification;
*: phosphorothioate (PS) bond;
“N” may be any natural or non-natural nucleotide.

The amino acid sequences of Cas effector proteins with nuclear localization signals (NLSs) and FLAG-tagged sequence are shown in SEQ ID NOs: 521-597, Table 7. And the corresponding DNA sequences encoding these proteins are SEQ ID NOs: 621-697.

TABLE 7
The amino acid sequences of Cas effector
proteins with NLSs and Flag

| Name | SEQ ID NO |
|---|---|
| GEBx0462 with | 521 |
| NLS and Flag | |
| GEBx0463 with | 522 |
| NLS and Flag | |
| GEBx0464 with | 523 |
| NLS and Flag | |
| GEBx0465 with | 524 |
| NLS and Flag | |
| GEBx0466 with | 525 |
| NLS and Flag | |
| GEBx0467 with | 526 |
| NLS and Flag | |
| GEBx0468 with | 527 |
| NLS and Flag | |
| GEBx0469 with | 528 |
| NLS and Flag | |
| GEBx0470 with | 529 |
| NLS and Flag | |
| GEBx0471 with | 530 |
| NLS and Flag | |
| GEBx0472 with | 531 |
| NLS and Flag | |
| GEBx0473 with | 532 |
| NLS and Flag | |
| GEBx0474 with | 533 |
| NLS and Flag | |
| GEBx0475 with | 534 |
| NLS and Flag | |
| GEBx0476 with | 535 |
| NLS and Flag | |
| GEBx0477 with | 536 |
| NLS and Flag | |
| GEBx0478 with | 537 |
| NLS and Flag | |
| GEBx0479 with | 538 |
| NLS and Flag | |
| GEBx0480 with | 539 |
| NLS and Flag | |
| GEBx0481 with | 540 |
| NLS and Flag | |
| GEBx0482 with | 541 |
| NLS and Flag | |
| GEBx0483 with | 542 |
| NLS and Flag | |
| GEBx0484 with | 543 |
| NLS and Flag | |
| GEBx0485 with | 544 |
| NLS and Flag | |
| GEBx0486 with | 545 |
| NLS and Flag | |
| GEBx0487 with | 546 |
| NLS and Flag | |
| GEBx0488 with | 547 |
| NLS and Flag | |
| GEBx0489 with | 548 |
| NLS and Flag | |
| GEBx0490 with | 549 |
| NLS and Flag | |
| GEBx0491 with | 550 |
| NLS and Flag | |
| GEBx0492 with | 551 |
| NLS and Flag | |
| GEBx0493 with | 552 |
| NLS and Flag | |
| GEBx0494 with | 553 |
| NLS and Flag | |
| GEBx0495 with | 554 |
| NLS and Flag | |
| GEBx0496 with | 555 |
| NLS and Flag | |
| GEBx0497 with | 556 |
| NLS and Flag | |
| GEBx0498 with | 557 |
| NLS and Flag | |
| GEBx0499 with | 558 |
| NLS and Flag | |
| GEBx0500 with | 559 |
| NLS and Flag | |
| GEBx0501 with | 560 |
| NLS and Flag | |
| GEBx0502 with | 561 |
| NLS and Flag | |
| GEBx0503 with | 562 |
| NLS and Flag | |
| GEBx0504 with | 563 |
| NLS and Flag | |
| GEBx0505 with | 564 |
| NLS and Flag | |
| GEBx0506 with | 565 |
| NLS and Flag | |
| GEBx0507 with | 566 |
| NLS and Flag | |
| GEBx0508 with | 567 |
| NLS and Flag | |
| GEBx0509 with | 568 |
| NLS and Flag | |
| GEBx0510 with | 569 |
| NLS and Flag | |
| GEBx0511 with | 570 |
| NLS and Flag | |
| GEBx0512 with | 571 |
| NLS and Flag | |
| GEBx0513 with | 572 |
| NLS and Flag | |
| GEBx0514 with | 573 |
| NLS and Flag | |
| GEBx0515 with | 574 |
| NLS and Flag | |
| GEBx0516 with | 575 |
| NLS and Flag | |
| GEBx0517 with | 576 |
| NLS and Flag | |
| GEBx0518 with | 577 |
| NLS and Flag | |
| GEBx0519 with | 578 |
| NLS and Flag | |
| GEBx0520 with | 579 |
| NLS and Flag | |
| GEBx0521 with | 580 |
| NLS and Flag | |
| GEBx0522 with | 581 |
| NLS and Flag | |
| GEBx0523 with | 582 |
| NLS and Flag | |
| GEBx0524 with | 583 |
| NLS and Flag | |
| GEBx0525 with | 584 |
| NLS and Flag | |
| GEBx0526 with | 585 |
| NLS and Flag | |
| GEBx0527 with | 586 |
| NLS and Flag | |
| GEBx0528 with | 587 |
| NLS and Flag | |
| GEBx0529 with | 588 |
| NLS and Flag | |
| GEBx0530 with | 589 |
| NLS and Flag | |
| GEBx0531 with | 590 |
| NLS and Flag | |
| GEBx0532 with | 591 |
| NLS and Flag | |
| GEBx0533 with | 592 |
| NLS and Flag | |
| GEBx0534 with | 593 |
| NLS and Flag | |
| GEBx0535 with | 594 |
| NLS and Flag | |
| GEBx0536 with | 595 |
| NLS and Flag | |
| GEBx0537 with | 596 |
| NLS and Flag | |
| GEBx0538 with | 597 |
| NLS and Flag | |

### Example 4: In Vitro Gene Editing Effect of the Cas Proteins in Mammalian Cell Line

In another set of experiment, HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 250 μL of cells with a density of 100,000 cells/well was mixed with 1 μL mixture of Lipofectamine™ 3000 (ThermoFisher Scientific, Cat. L3000008), 2 μL P3000, Opti-Mem (Volume refill to 25 μL), and pCasX plasmid (375 ng) and psgRNA plasmid encoding a sgRNA targeting endogenous gene (125 ng) per the manufacturer's protocol. Then seeded the cell mixture onto a 48-well plate and cultured at 37° C. and 5% CO₂.

72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples (500 ng, 260/280 value: 1.8~2.0) were subjected to amplicons NGS analyses.

To quantitatively determine the efficiency of editing at the target location in the genome, NGS was utilized to identify the presence of insertions and deletions introduced by gene editing. Primers used for NGS which around the target area within the endogenous genes were designed. Additional PCR was performed per the manufacturer's protocols (Illumina) to add chemistry for sequencing. The amplicons were sequenced on Illumina iSeq 100. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected and the number of wild types reads versus the number of reads which contain an insertion, substitution, or deletion was calculated. The number of the reads mapped the reference genome is more than 1000.

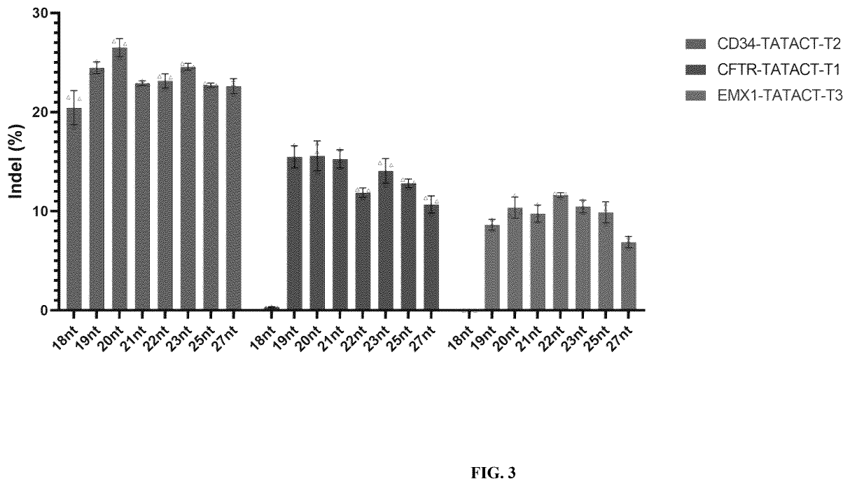

In an in vitro experiment using HEK293T cells, we evaluated the efficacy of GEBx0530 on three endogenous targets across a range of spacer lengths. The pCasX plasmid containing sequences encoding GEBx0530 (SEQ ID NO: 589) was co-transfected with psgRNA plasmid harboring a sequence encoding sgRNA with different-length spacers (SEQ ID NOs: 901-924, Table 8) encoding sequences. As shown in FIG. 3, Cas effector protein GEBx0530 exhibited editing activity on all three targets, with peak efficiencies of 26.51% (CD34-TATACT-T2-20nt), 15.59% (CFTR-TATACT-T1-20nt), and 11.64% (EMX1-TATACT-T3-22nt), respectively.

TABLE 8
The nucleotide sequence of the spacer described in this example

| | SEQ ID | |
|---|---|---|
| Name | NO: | 5′ to 3′ |
| CD34-TATACT-T2-18 nt | 901 | AGGCACCCGGTAAATATT |
| CD34-TATACT-T2-19 nt | 902 | TAGGCACCCGGTAAATATT |
| CD34-TATACT-T2-20 nt | 903 | aTAGGCACCCGGTAAATATT |
| CD34-TATACT-T2-21 nt | 904 | aaTAGGCACCCGGTAAATATT |
| CD34-TATACT-T2-22 nt | 905 | aaaTAGGCACCCGGTAAATATT |
| CD34-TATACT-T2-23 nt | 906 | aaaaTAGGCACCCGGTAAATATT |
| CD34-TATACT-T2-25 nt | 907 | aaaaaaTAGGCACCCGGTAAATATT |
| CD34-TATACT-T2-27 nt | 908 | aaaaaaaaTAGGCACCCGGTAAATATT |
| CFTR-TATACT-T1-18 nt | 909 | GTGAAAGAATGACTCTCT |
| CFTR-TATACT-T1-19 nt | 910 | AGTGAAAGAATGACTCTCT |
| CFTR-TATACT-T1-20 nt | 911 | GAGTGAAAGAATGACTCTCT |
| CFTR-TATACT-T1-21 nt | 912 | GGAGTGAAAGAATGACTCTCT |
| CFTR-TATACT-T1-22 nt | 913 | AGGAGTGAAAGAATGACTCTCT |
| CFTR-TATACT-T1-23 nt | 914 | TAGGAGTGAAAGAATGACTCTCT |
| CFTR-TATACT-T1-25 nt | 915 | CCTAGGAGTGAAAGAATGACTCTCT |
| CFTR-TATACT-T1-27 nt | 916 | GGCCTAGGAGTGAAAGAATGACTCTCT |
| EMX1-TATACT-T3-18 nt | 917 | CATTACCAGACATATGAA |
| EMX1-TATACT-T3-19 nt | 918 | CCATTACCAGACATATGAA |
| EMX1-TATACT-T3-20 nt | 919 | ACCATTACCAGACATATGAA |
| EMX1-TATACT-T3-21 nt | 920 | AACCATTACCAGACATATGAA |
| EMX1-TATACT-T3-22 nt | 921 | TAACCATTACCAGACATATGAA |
| EMX1-TATACT-T3-23 nt | 922 | ATAACCATTACCAGACATATGAA |
| EMX1-TATACT-T3-25 nt | 923 | AGATAACCATTACCAGACATATGAA |
| EMX1-TATACT-T3-27 nt | 924 | ATAGATAACCATTACCAGACATATGAA |

### Example 5: Structure-Guide Engineering of the GEBx0530 for PAM Expansion and Efficiency Improvement

In the realm of genome editing, the necessity for PAM recognition constrains the targeting resolution of CRISPR and renders certain genomic loci inaccessible for modification. To broaden the spectrum of PAMs available to CRISPR enzymes and enhance gene editing efficiency, structure-guided engineering was employed to generate additional variants of GEBx0530.

The prediction of the GEBx0530 RNP structure was performed using AlphaFold 3 (Josh et al., 2024, Nature). The sequences of DNA and RNA are presented in Table 9.

TABLE 9
The DNA and RNA used for prediction of the GEBx0530 RNP
structure

| | SEQ ID | | |
|---|---|---|---|
| Name | NOs: | Type | 5′-3′ |
| TS | 704 | DNA | CAAAGTACCTAGAGAGTGCCTTGCAGTGAATAG |
| DNA | | | |
| NTS | 705 | DNA | TAGGTACTTT |
| DNA | | | |
| GEBx0530- | 706 | RNA | UUCACUGCAAGGCACUCUCUAGUUUGAGAAUGG |
| HPT-V1 | | | UGUAAUUCUGUAUGUAGUCAAGAAAUUGACUAC |
| sgRNA | | | CAUACAAGAUUACACCAUAAGUUCAAAUAAAGA |
| | | | UUUUAUCCAAAUCGUCAUGUUUACAUGACCCCA |
| | | | CAGUGUGUGGAUUAAACUUGCUUCGGCAAGUUU |
| | | | UU |

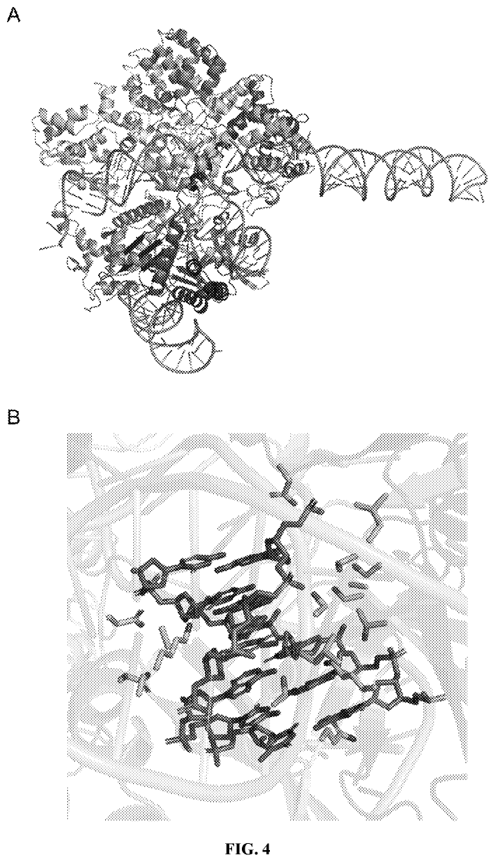

FIG. 4A presents a rendering of the predicted RNP structure of GEBx0530. As illustrated in FIG. 4B, 18 residues (A1112, A1113, A1309, D1080, D931, I1193, K1111, K1315, L1298, N1110, Q1191, S1185, S1297, S1299, S1310, S1310, T1188, T1313) within the CTD domains surrounding the PAM binding site of GEBx0530 were selected for subsequent mutant research.

### Example 6: PAM Determination of GEBx0530 Variants in Mammalian Cell Line

In a set of experiments, HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 450 μL of cells with a density of 120,000 cells/well was mixed with 50 μL mixture containing Lipofectamine™ 3000 (ThermoFisher Scientific, Cat. L3000008), Opti-Mem (Volume refill to 50 μL), 1 μL dsODN (2.5 pM), 100 ng (~1 μL) psgRNA harbored sgRNA scaffold (SEQ ID NO: 841, Table 16) encoding sequence and Humanspacer3 spacer (SEQ ID NO: 500) encoding sequence and 400 ng (~1 μL) pCasX plasmid harbored CDS encoding Cas proteins with NLS and FLAG (Table 10) per the manufacturer's protocol. Then seeded the cell mixture onto a 24-well plate and cultured at 37° C. and 5% CO₂. 10 pM dsODN was annealed using dsODN-Top and dsODN-BOT oligonucleotides pre-transfection.

72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples (500 ng, 260/280 value: 1.8~2.0) were subjected to Guide-Seq NGS analyses.

The basic method of Guide-Seq library preparation is described by Nikolay et. al (Nat. Protoc. 2021). The extracted DNA sample were first sheared using KAPA Frag Kit (Cat #KK8602, Roche). Fragmented DNA was purified and then phosphorated using T4 Polynucleotide Kinase (Cat #M0201S, NEB). An SS5-adapter (generated by annealing 10 μM SS5TOP oligo with 10 μM SS5B™ oligo) was ligated to the fragmented DNA using Quick Ligation™ Kit (Cat #M2200S, NEB), followed by two steps off-target PCR to add chemistry for sequencing.

For off-target PCR1 was performed using Platinum™ Taq DNA Polymerase (Cat #15966005, Invitrogen) with GSP1 (a mixture of GSP1-Top and GSP1-BoT) and Y_XX oligos. For off-target PCR2 was performed using Platinum™ Taq DNA Polymerase with GSP2 (a mixture of GSP2-TopA/B/C and GSP1-BoTA/B/C), Y_XX (Same to PCR1) and i753_XX oligos. The DNA product in each step described above need purification using SPRI Select (Cat #B23318, Beckman Coulter). The final library was quantified with qPCR and sequenced on Illumina NextSeq 1000. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected.

The amino acid sequences of Cas proteins with nuclear localization signals (NLSs) and FLAG-tagged sequence are shown in SEQ ID NOs: 589, 711-725 (Table 10). And the corresponding DNA sequences encoding these proteins are SEQ ID NOs: 689, 731-745.

TABLE 10
The Cas protein sequences with NLS and Flag

| Name | SEQ ID NOs: |
|---|---|
| GEB 530-wt | 589 |
| with NLS and Flag | |
| GEBx0530-K1315Q | 711 |
| with NLS and Flag | |
| GEBx0530-K1315N | 712 |
| with NLS and Flag | |
| GEBx0530-K1315T | 713 |
| with NLS and Flag | |
| GEBx0530-A1113N | 714 |
| with NLS and Flag | |
| GEBx0530-A1113K | 715 |
| with NLS and Flag | |
| GEBx0530-A1113R | 716 |
| with NLS and Flag | |
| GEBx0530-Q1191N | 717 |
| with NLS and Flag | |
| GEBx0530-Q1191K | 718 |
| with NLS and Flag | |
| GEBx0530-Q1191V | 719 |
| with NLS and Flag | |
| GEBx0530-L1298R | 720 |
| with NLS and Flag | |
| GEBx0530-DM3(A1113R/ | 721 |
| K1315Q) with NLS and | |
| Flag | |
| GEBx0530-DM6(L1298R/ | 722 |
| K1315Q) with NLS and | |
| Flag | |
| GEBx0530-DM7(A1113R/ | 723 |
| K1315T) with NLS and | |
| Flag | |
| GEBx0530-TM1(A1113R/ | 724 |
| L1298R/K1315Q) with | |
| NLS and Flag | |
| GEBx0530-TM2(A1113R/ | 725 |
| L1298R/K1315T) with | |
| NLS and Flag | |

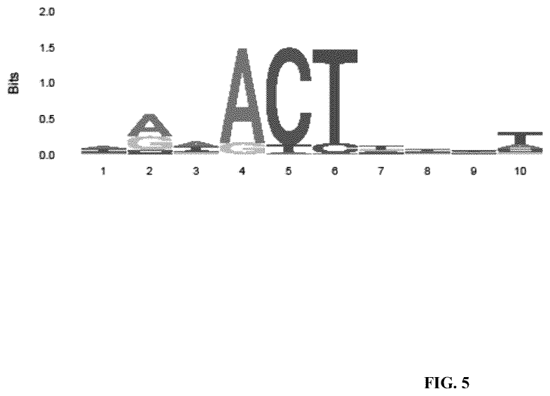

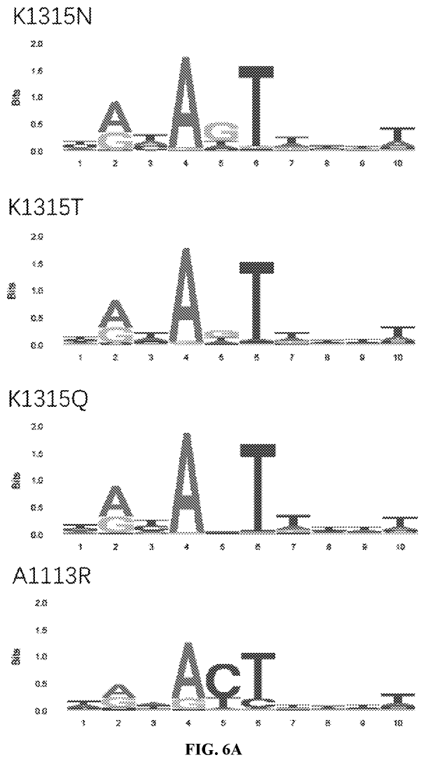

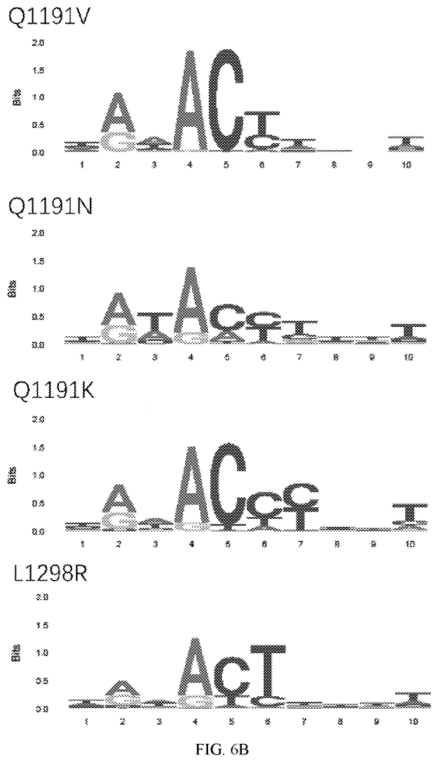

As illustrated in FIG. 5, FIG. 6A and FIG. 6B, the GEBx0530-K1315N/T/Q and Q1191V/N/K variants significantly modify positions 5 to 7 of the PAM (altering from NRNACT to NRNAGT/NRNANT or NRNACY/NRNACYY). Additionally, certain mutants (such as GEBx0530-A1113R or GEBx0530-L1298R) do not change the preference of PAM but enhance the activity of the Cas protein (quantified by the number of editing sites, Table 11).

TABLE 11
Unique read count and Num of sites of the Cas effector proteins

| Name | Unique read count | Num of sites |
|---|---|---|
| GEBx0530-WT | 2203593 | 424 |
| GEBx0530-A1113R | 882990 | 513 |
| GEBx0530-L1298R | 1799652 | 752 |

### Example 7: In Vitro Gene Editing Effect of GEBx0530 in Mammalian Cell Line

In a set of experiments, the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 250 μL of cells with a density of 50,000 cells/well was seeded onto a 48-well plate 24 hours pre-transfection. Cells were transfected with a lipoplex containing Lipofectamine™ 3000 (0.4 μL/well), P3000 (2 μL/well), pgRNA/pCasX plasmid (125 ng/well and 375 ng/well, respectively) and Opti-Mem up to 25 μL/well per the manufacturer's protocol. Plated cells were allowed to settle and adhere for 72 hours in a tissue culture incubator at 37° C. and 5% CO₂ atmosphere. The nucleotide sequences of the pgRNA used in this example are composed of the sgRNA scaffold GEBx0530-HPT-V1 (SEQ ID NO: 841) encoding sequence and the corresponding spacers (SEQ ID NO: 761-776, Table 12) encoding sequences.

TABLE 12
The spacer sequences in the examples

| Name | SEQ ID NOs | Sequence |
|---|---|---|
| CD34-TATACT-T1 | 761 | CCAGCAATCCGCTCCGTGTC |
| CD34-TATACT-T2 | 762 | Ataggcacccggtaaatatt |
| CD34-TATACT-T3 | 763 | CCACAGACCCTTGGTTTATA |
| CD34-TATACT-T6 | 764 | TTTGGTCACTCTCCATCCAG |
| CFTR-TATACT-T1 | 765 | GAGTGAAAGAATGACTCTCT |
| CFTR-TATACT-T3 | 766 | GACTCATTTGGCTACAGTGC |
| CFTR-TATACT-T5 | 767 | CAGTATTCTGGGGTAAGCTC |
| CFTR-TATACT-T7 | 768 | GTGTGCTAATTTATATGCTC |
| EMX1-TATACT-T3 | 769 | ACCATTACCAGACATATGAA |
| EMX1-TATACT-T4 | 770 | TCTACTTCTCTGTGTTTCTG |
| POLQ-TATACT-T1 | 771 | CAGGCAGTCGTACTACAAGG |
| POLQ-TATACT-T2 | 772 | ATGTGTGTGTATACATAGTC |
| POLQ-TATACT-T3 | 773 | GGCACCAAGCACCATCATCC |
| POLQ-TATACT-T4 | 774 | GTTGCAGGAACCTGTACATA |
| POLQ-TATACT-T5 | 775 | tgagttgcatggggcatact |
| POLQ-TATACT-T6 | 776 | TAGCAAAGCCATTTCATGAA |

72 hours post-transfection, the supernatant was removed and the cell layer was washed by PBS. Then the genomic DNA was extracted from each well of a 24-well plate using DNA Extraction solution (Denogen (Beijing) Bio Sci & Tech Co. Ltd, Cat. DNS033-48) per manufacturer's protocol. All DNA samples (500 ng, 260/280 value: 1.8~2.0) were subjected to amplicons NGS analyses.

To quantitatively determine the efficiency of editing at the target location in the genome, NGS was utilized to identify the presence of insertions and deletions introduced by gene editing. Primers used for NGS which around the target area within the endogenous genes were designed. Additional PCR was performed per the manufacturer's protocols (Illumina) to add chemistry for sequencing. The amplicons were sequenced on Illumina iSeq 100. The reads were aligned to a reference genome after eliminating those having low quality scores. Q30 rate is more than 0.9. The reads length is between 130 bp-140 bp. The resulting files containing the reads were mapped to the reference genome (BAM files), where reads that overlapped the target region of interest were selected and the number of wild types reads versus the number of reads which contain an insertion, substitution, or deletion was calculated. The number of the reads mapped the reference genome is more than 1000.

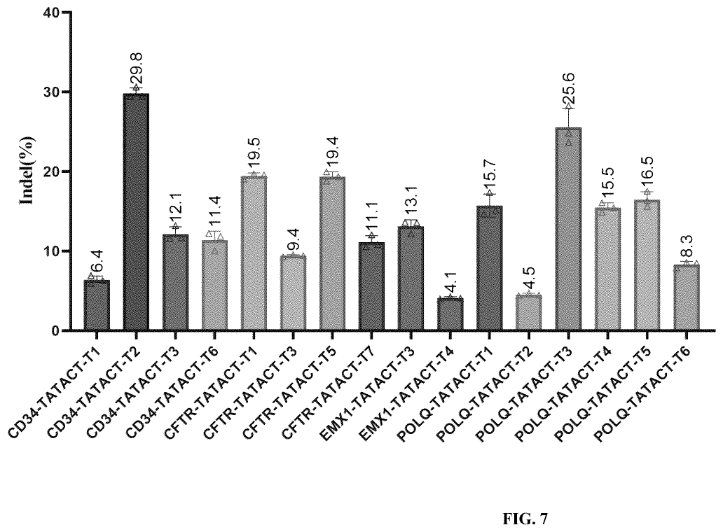

In an in vitro experiment, GEBx0530 was tested on 16 endogenous targets in the HEK293T cell line. The pCasX plasmid containing the GEBx0530 CDS was co-transfected with pgRNA plasmid harboring corresponding spacers (SEQ ID NOs: 761-776) encoding sequences. The results, shown in FIG. 7, demonstrate an indel activity ranging from 4.1% to 29.8%.

### Example 8: Combination Mutations Involving Multiple Amino Acid Residues in GEBx0530

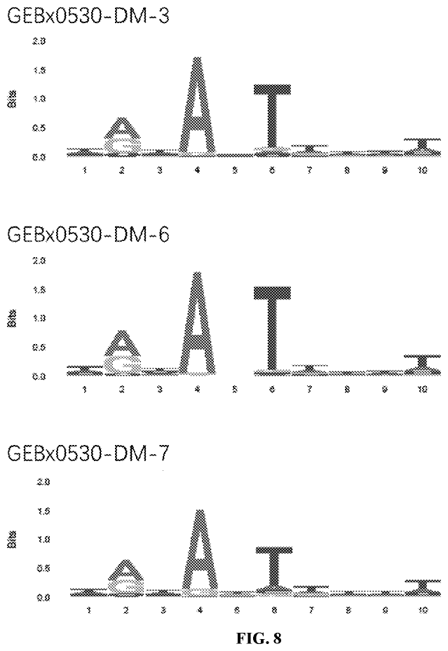

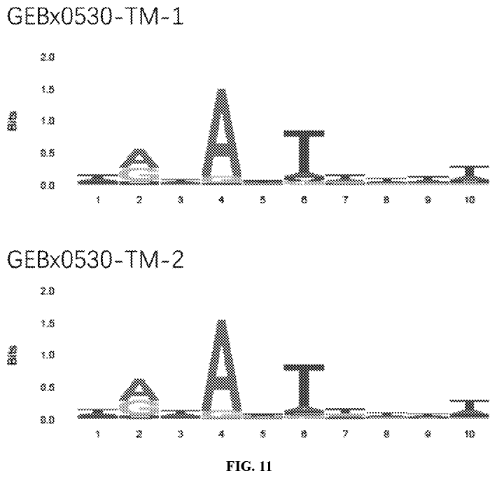

To further optimize the function of GEBx0530, we combined the mutation sites selected in Example 6 to generate GEBx0530 variants with double or triple mutations (shown in Table 13). The method for determining the PAM of GEBx0530 mutants is described in the same manner as in Example 6. The result is illustrated in FIG. 8 and FIG. 11. Both the GEBx0530-DM-3/7 and GEBx0530-TM-1/2 mutants retained the PAM expansion introduced by the K1315 site mutation, while also exhibiting a significant enhancement in editing activity (quantified by the number of editing sites, Table 14).

TABLE 13
Types of mutations in GEBx0530 variants.

| Name | Type of mutations |
|---|---|
| GEBx0530-DM3 | A1113R/K1315Q |
| GEBx0530-DM6 | L1298R/K1315Q |
| GEBx0530-DM7 | A1113R/K1315T |
| GEBx0530-TM1 | A1113R/L1298R/K1315Q |
| GEBx0530-TM2 | A1113R/L1298R/K1315T |

TABLE 14
Unique read count and Num of sites of the Cas proteins

| Name | Unique read count | Num of sites |
|---|---|---|
| GEBx0530-DM-3 | 1038617 | 1173 |
| GEBx0530-DM-6 | 1494811 | 653 |
| GEBx0530-DM-7 | 3348113 | 2221 |
| GEBx0530-TM-1 | 2070511 | 2931 |
| GEBx0530-TM-2 | 1130187 | 1722 |

In another in vitro experiments, the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 250 μL of cells with a density of 50,000 cells/well was seeded onto a 48-well plate 24 hours pre-transfection. Cells were transfected with a lipoplex containing Lipofectamine™ 3000 (0.4 μL/well), P3000 (2 μL/well), pgRNA/pCasX-GEBx0530-DM3 plasmid (125 ng/well and 375 ng/well, respectively) and Opti-Mem up to 25 μL/well per the manufacturer's protocol. Plated cells were allowed to settle and adhere for 72 hours in a tissue culture incubator at 37° C. and 5% CO₂ atmosphere. The nucleotide sequences of the pgRNA used in this example are composed of the sgRNA scaffold GEBx0530-HPT-V1 (SEQ ID NO: 841) encoding sequence and the spacers (SEQ ID NOs: 781-804, Table 15) encoding sequence with NATANT PAM. Genomic DNA extraction and NGS amplicon sequencing are described in the same manner as in Example 7.

TABLE 15
The exemplary spacer sequences referred to in this example

| Name | SEQ ID NOs | Sequences |
|---|---|---|
| RFN2-NATAAT-T1 | 781 | TAGCCAGGATCAACAAGCAC |
| RFN2-NATAAT-T2 | 782 | AAAATGGTAGTGGAGCAGAA |
| RFN2-NATAAT-T3 | 783 | ATGATTCTGGGCTAGAGCTT |
| RFN2-NATAAT-T4 | 784 | TTCCCCATTAAAATACCAGA |
| RFN2-NATAAT-T5 | 785 | GATGTGGCAACCCAAAATGA |
| RFN2-NATAAT-T6 | 786 | ATATTCAGTGGGCATGCATT |
| RFN2-NATACT-T1 | 787 | TAAAGCTAACCTCACAGCCA |
| RFN2-NATACT-T2 | 788 | GGCTGTTGCTATATAAATGG |
| RFN2-NATACT-T3 | 789 | GAGCCTTGGGTCTTCATGAA |
| RFN2-NATACT-T4 | 790 | TGATGCAGTTTATATCCTCC |
| RFN2-NATACT-T5 | 791 | TTTGGAATTGGTCAGTGAGA |
| RFN2-NATACT-T6 | 792 | GTACAGAAGAGGGGAAAGTA |
| RFN2-NATAGT-T1 | 793 | CATTCTCTAAGCACAAAGCT |
| RFN2-NATAGT-T2 | 794 | GGGAGAAGACCTTAGGAAAG |
| RFN2-NATAGT-T3 | 795 | CATAAGGAGGACACGTTTCA |
| RFN2-NATAGT-T4 | 796 | AGTAGTGTTAGAGGAGATGA |
| RFN2-NATAGT-T5 | 797 | TCCTTCCAAGGTCATTAGAA |
| RFN2-NATAGT-T6 | 798 | ACACCTACGCTCTTAGCACC |
| RFN2-NATATT-T1 | 799 | TGCGGAAGCTGAGGCTCGCC |
| RFN2-NATATT-T2 | 800 | ATGAGAACTTGGTTCATTGA |
| RFN2-NATATT-T3 | 801 | TCAAACGTATACTTGAAGAC |
| RFN2-NATATT-T4 | 802 | AAAATACCAGAGATAATGGA |
| RFN2-NATATT-T5 | 803 | ATCTCTCTCCAGACTCTAGG |
| RFN2-NATATT-T6 | 804 | AAGTCTGTCTAGTCATTGTA |

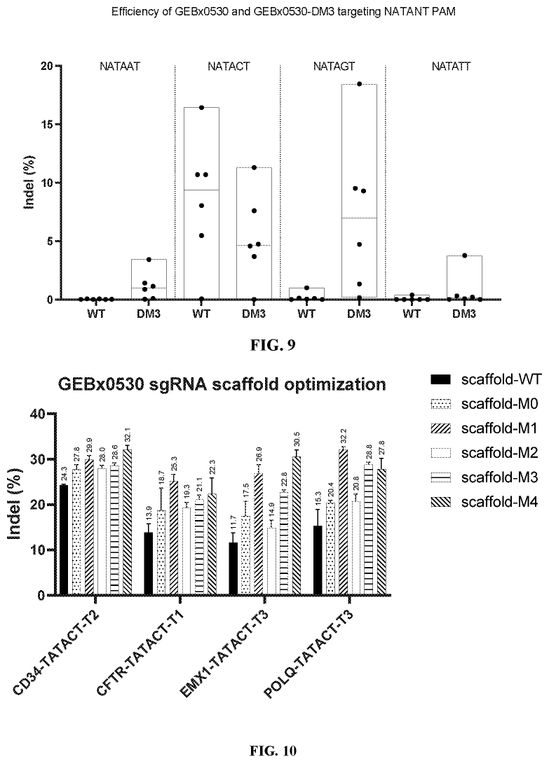

The result presented in FIG. 9 indicates that GEBx0530-WT is exclusively highly sensitive to NATACT PAM, whereas GEBx0530-DM3 exhibits activity against all four PAMs (NATACT/NATAGT/NATAAT/NATATT), thereby demonstrating the conversion of the fifth position from C to N in PAM.

### Example 9: sgRNA Modification of GEBx0530

To further enhance the gene editing efficiency of GEBx0530, various sgRNA scaffolds were evaluated. In a set of experiments, the HEK293T cells were cultured in DMEM media supplemented with 10% fetal bovine serum (Gibco™). For reverse transfection, the HEK293T cells were cultured in DMEM media supplemented with 10% fetal bovine serum (Gibco™). A volume of 250 μL of cells with a density of 50,000 cells/well was seeded onto a 48-well plate 24 hours pre-transfection. Cells were transfected with a lipoplex containing Lipofectamine™ 3000 (0.4 μL/well), P3000 (2 μL/well), pgRNA and pCasX plasmid (125 ng/well and 375 ng/well, respectively), and Opti-Mem up to 25 μL/well per the manufacturer's protocol. Plated cells were allowed to settle and adhere for 72 hours in a tissue culture incubator at 37° C. and 5% CO₂ atmosphere. The nucleotide sequences of the pgRNA used in this example are composed of the different sgRNA scaffold (SEQ ID NOs: 841-846, Table 16) encoding sequences and the some specific spacers (SEQ ID NOs: 762, 765, 769, 773, Table 12) encoding sequences. Genomic DNA extraction and NGS amplicon sequencing are described in the same manner as in Example 7.

TABLE 16
The gRNA and their fragments in this example

| Name | SEQ ID NOs | Sequences |
|---|---|---|
| GEBx0530- | 821 | UUGACUACCAUACAAGAUUACACCAUAAGUUC |
| HPT-V1 (WT)- | | AAAUAAAGAUUUUAUCCAAAUCGUCAUGUUUA |
| Tracr- | | CAUGACCCCACAGUGUGUGGAUUAAACUUGCU |
| | | UCGGCAAGUUUUU |
| GEBx530- | 822 | UACACCAUAAGUUCAAAUAAAGAUUUUAUCCA |
| HPT-V1-M0- | | AAUCGUCAUGUUUACAUGACCCCACAGUGUGU |
| Tracr | | GGAUUAAACUUGCUUCGGCAAGUUUU |
| GEBx530- | 823 | UACACCAUAAGUUCAAAUAAAGAUcUUAUCCAg |
| HPT-V1-M1- | | AUCGUCAUGUUUACAUGACCCCACAGUGUGUG |
| Tracr | | GAUUAAACUUGCUUCGGCAAGUUUU |
| GEBx530- | 824 | UACACCAUAAGUUCAAAUAAAGAUUUUAUCCA |
| HPT-V1-M2- | | AAUCGUCAUGUUUACAUGACCCCACgacAGUGU |
| Tracr | | gucGUGGAUUAAACUUGCUUCGGCAAGUUUU |
| GEBx530- | 825 | UACACCAUAAGUUCAAAUAAAGAUcUUAUCCAg |
| HPT-V1-M3- | | AUCGUCAUGUUUACAUGACCCCACgacAGUGUgu |
| Tracr | | cGUGGAUUAAACUUGCUUCGGCAAGUUUU |
| GEBx530- | 826 | UACACCAUAAGUUCAAAUAAAGAUUUUAUCCA |
| HPT-V1-M4- | | AAUCGUCAUGUUUACAUGACCCCACAGUGUGU |
| Tracr | | GGAUU |
| GEBx0530- | 841 | GUUUGAGAAUGGUGUAAUUCUGUAUGUAGUCA |
| HPT-V1 (WT) | | AGAAAUUGACUACCAUACAAGAUUACACCAUA |
| Scaffold | | AGUUCAAAUAAAGAUUUUAUCCAAAUCGUCAU |
| | | GUUUACAUGACCCCACAGUGUGUGGAUUAAAC |
| | | UUGCUUCGGCAAGUUUUU |
| GEBx530- | 842 | GUUUGAGAAUGGUGUAgaaaUACACCAUAAGUU |
| HPT-V1-M0 | | CAAAUAAAGAUUUUAUCCAAAUCGUCAUGUUU |
| Scaffold | | ACAUGACCCCACAGUGUGUGGAUUAAACUUGC |
| | | UUCGGCAAGUUUU |
| GEBx530- | 843 | GUUUGAGAAUGGUGUAgaaaUACACCAUAAGUU |
| HPT-V1-M1 | | CAAAUAAAGAUcUUAUCCAgAUCGUCAUGUUUA |
| Scaffold | | CAUGACCCCACAGUGUGUGGAUUAAACUUGCU |
| | | UCGGCAAGUUUU |
| GEBx530- | 844 | GUUUGAGAAUGGUGUAgaaaUACACCAUAAGUU |
| HPT-V1-M2 | | CAAAUAAAGAUUUUAUCCAAAUCGUCAUGUUU |
| Scaffold | | ACAUGACCCCACgacAGUGUgucGUGGAUUAAAC |
| | | UUGCUUCGGCAAGUUUU |
| GEBx530- | 845 | GUUUGAGAAUGGUGUAgaaaUACACCAUAAGUU |
| HPT-V1-M3 | | CAAAUAAAGAUcUUAUCCAgAUCGUCAUGUUUA |
| Scaffold | | CAUGACCCCACgacAGUGUgucGUGGAUUAAACU |
| | | UGCUUCGGCAAGUUUU |
| GEBx530- | 846 | GUUUGAGAAUGGUGUAgaaaUACACCAUAAGUU |
| HPT-V1-M4 | | CAAAUAAAGAUUUUAUCCAAAUCGUCAUGUUU |
| Scaffold | | ACAUGACCCCACAGUGUGUGGAUU |
| GEBx0530- | 861 | NNNNNNNNNNNNNNNNNNNNGUUUGAGAAUG |
| HPT-V1 (WT) | | GUGUAAUUCUGUAUGUAGUCAAGAAAUUGACU |
| | | ACCAUACAAGAUUACACCAUAAGUUCAAAUAA |
| | | AGAUUUUAUCCAAAUCGUCAUGUUUACAUGAC |
| | | CCCACAGUGUGUGGAUUAAACUUGCUUCGGCA |
| | | AGUUUUU |
| GEBx530- | 862 | NNNNNNNNNNNNNNNNNNNNGUUUGAGAAUG |
| HPT-V1-M0 | | GUGUAgaaaUACACCAUAAGUUCAAAUAAAGAU |
| | | UUUAUCCAAAUCGUCAUGUUUACAUGACCCCA |
| | | CAGUGUGUGGAUUAAACUUGCUUCGGCAAGUU |
| | | UU |
| GEBx530- | 863 | NNNNNNNNNNNNNNNNNNNNGUUUGAGAAUG |
| HPT-V1-M1 | | GUGUAgaaaUACACCAUAAGUUCAAAUAAAGAUc |
| | | UUAUCCAgAUCGUCAUGUUUACAUGACCCCACA |
| | | GUGUGUGGAUUAAACUUGCUUCGGCAAGUUUU |
| GEBx530- | 864 | NNNNNNNNNNNNNNNNNNNNGUUUGAGAAUG |
| HPT-V1-M2 | | GUGUAgaaaUACACCAUAAGUUCAAAUAAAGAU |
| | | UUUAUCCAAAUCGUCAUGUUUACAUGACCCCA |
| | | CgacAGUGUgucGUGGAUUAAACUUGCUUCGGCA |
| | | AGUUUU |
| GEBx530- | 865 | NNNNNNNNNNNNNNNNNNNNGUUUGAGAAUG |
| HPT-V1-M3 | | GUGUAgaaaUACACCAUAAGUUCAAAUAAAGAUc |
| | | UUAUCCAgAUCGUCAUGUUUACAUGACCCCACg |
| | | acAGUGUgucGUGGAUUAAACUUGCUUCGGCAAG |
| | | UUUU |
| GEBx530- | 866 | NNNNNNNNNNNNNNNNNNNNGUUUGAGAAUG |
| HPT-V1-M4 | | GUGUAgaaaUACACCAUAAGUUCAAAUAAAGAU |
| | | UUUAUCCAAAUCGUCAUGUUUACAUGACCCCA |
| | | CAGUGUGUGGAUU |

“N” denotes any natural or non-natural nucleotide. The consecutive ‘N’s depicted represent the corresponding sequence of the spacer region. It is important to note that the number of ‘n’s shown is illustrative only and does not impose a limitation on the contiguous sequence of ‘N’s. This consecutive ‘N’s sequence may also denote either a greater (e.g., 15, 16, 17, 18, 19) or fewer (e.g., 21, 22, 23, 24, 25, 26) number of nucleotides than presented, depending on the specific design requirements of the RNA.

FIG. 10 illustrates the indel levels of GEBx0530 targeting four endogenous genes using modified RNA scaffolds. The sgRNA sequences employed in this experiment included GEBx530-HPT-V1 (WT, SEQ ID NO: 861) and GEBx530-HPT-V1-M0 to GEBx530-HPT-V1-M4 (M0-M4, SEQ ID NOs: 862-866). The GEBx530-M1 and M4 scaffolds exhibited the two highest mean indel values.

### Example 10: In Vitro Gene Editing Using VLP-Delivered GEBx0530 Mutants

The VLP packaging procedure was initiated by seeding HEK293T cells at a density of 1.13×10⁶ cells per T25 flask in 6.5 mL DMEM supplemented with 10% FBS 48 hours prior to transfection, allowing cells to reach 80-90% confluency under standard culture conditions (37° C., 5% CO₂). Transfection complexes were prepared by combining plasmids containing envelope protein (VSVG) encoding sequences, MMLV Gag-pol encoding sequence, and MMLV Gag-GEBx0530-TM-1 encoding sequences and sgRNA (with GEBx530-HPT-V1-M4 scaffold and spacers selected from sequences as set forth in Table 17) encoding sequence. All the VLP packaging plasmids were dissolved in 166 μL Opti-MEM, followed by mixing with PEI MAX® (Polysciences 24765-1) dissolved in 166 μL Opti-MEM at a 3:1 (w/w) polymer: DNA ratio through dropwise addition and 10-second vertexing. After 10-minute incubation at room temperature for nanoparticle formation, the DNA-PEI complexes were administered to cells cultured in 3 mL fresh DMEM/10% FBS medium, with a 6-hour incubation prior to medium replacement with 6.5 mL serum-free DMEM. Supernatants were harvested 48 hours post-transfection through 0.45 μm PVDF filtration, precipitated overnight at 4° C. with 1.7 mL 50% (w/w) PEG8000 (Sigma 89510) under gentle agitation (50 rpm), and concentrated via centrifugation at 50,000 g for 2 hours (4° C.). The resultant VLP pellets were resuspended in 120 μL sterile PBS (pH 7.4) to generate 50× concentrated stocks, which were aliquoted and stored at −80° C. with ≤3 freeze-thaw cycles permitted.

In a set of experiments, the Hepa 1-6 cell were cultured in DMEM medium supplemented with 10% fetal bovine serum (Gibco™). For transfection, cells were seeded in a 96-well plate at 20,000 cells/well in 100 μL medium 24 hours prior. Each well received 2 μL (low dosage) or 10 μL (High dosage) of VLP 50× concentrated stocks. Following transfection, cells were maintained at 37° C. with 5% CO₂ for 72 hours. DNA extraction and Amplicon-Seq-based NGS analysis were performed as described in Example 7.

TABLE 17
Exemplary spacer sequences referred to in this
example

| Name | Spacer sequences | SEQ ID NOs |
|---|---|---|
| BS740 | aatcgtactggaagacactt | 881 |
| BS741 | gctgagcagggctgcgatgg | 882 |
| BS745 | cctgtgagcgatccagacat | 883 |
| BS746 | gaactagagtactcagactc | 884 |
| BS747 | tctgggggatctggttgatt | 885 |
| BS748 | agcatgaccaggaaccagag | 886 |
| BS749 | agcacttagcagagcagcta | 887 |
| BS750 | ggtggaaatggaaatcactt | 888 |
| BS753 | tagattccacagccagaatc | 889 |
| BS754 | taggttgatcataaggatga | 890 |
| BS756 | aatactccgcactcaaaggc | 891 |
| BS757 | tctcatctaatgctagaggc | 892 |
| BS758 | gttgctgcacccatgtctca | 893 |
| BS759 | gaggaccaggatcttgccaa | 894 |
| BS760 | ttactctgacccatttcact | 895 |

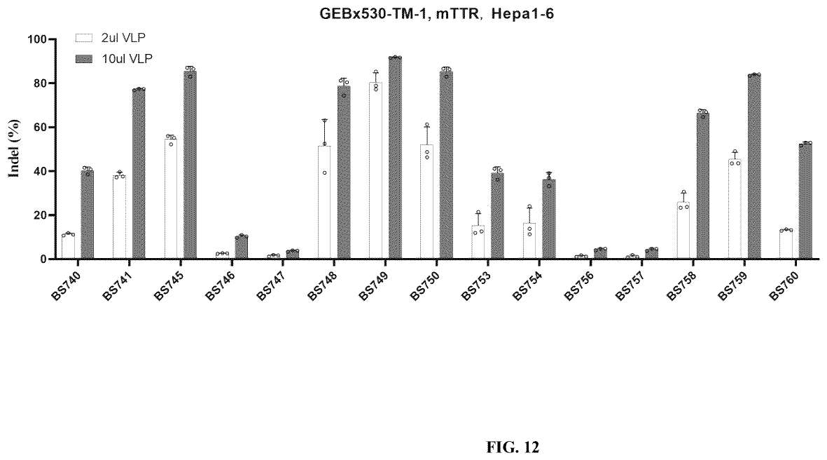

Assessment by deep sequencing revealed peak editing efficiencies of 80.47% (low dose) and 91.73% (high dose) at the mTTR locus (FIG. 12).

The CasX refers to the corresponding Cas proteins described herein, and the pCasX used in the examples refers to a plasmid that encodes such corresponding Cas proteins described herein.

The exemplary embodiments of the present disclosure are thus fully described. Although the description referred to particular embodiments, it will be clear to one skilled in the art that the present disclosure may be practiced with variation of these specific details. Hence this disclosure should not be construed as limited to the embodiments set forth herein.

1. An engineered, non-naturally occurring Type II CRISPR-associated (Cas) protein comprising:
(a) an amino acid sequence that has 100%, or at least 70%, at least 75%, at least 80%, at least 85%, at least 90%, at least 92%, at least 95%, at least 98%, or at least 99% sequence identity to an amino acid sequence of SEQ ID NO: 69; or
(b) an amino acid sequence that has 100%, or at least 70%, at least 75%, at least 80%, at least 85%, at least 90%, at least 92%, at least 95%, at least 98%, or at least 99% sequence identity to an amino acid sequence of SEQ ID NO: 69, with an exception of an amino acid “M” at position 1 of the amino acid sequence of SEQ ID NO: 69.

2. The engineered, non-naturally occurring Type II CRISPR-associated (Cas) protein of claim 1, comprising an amino acid mutation at one or more of the following positions: A1113, L1298, K1315 and Q1191.

3. The engineered, non-naturally occurring Type II Cas protein of claim 2, wherein the Cas protein comprises amino acid mutations at at least two positions of Q1191, A1113, L1298, and K1315.

4. The engineered, non-naturally occurring Type II Cas protein of claim 2, wherein the Cas protein comprises amino acid mutations at at least three positions of Q1191, A1113, L1298, and K1315; or, the Cas protein comprises amino acid mutations at the following positions: A1113, L1298 and K1315.

5. The engineered, non-naturally occurring Type II Cas protein of claim 2, wherein the Cas protein satisfies at least one of the following conditions:
(a) the mutation at position K1315 is selected from any one of K1315Q, K1315N and K1315T;
(b) the mutation at position A1113 is selected from any one of A1113N, A1113K and A1113R;
(c) the mutation at position Q1191 is selected from any one of Q1191N, Q1191K and Q1191V; and
(d) the mutation at position L1298 is L1298R.

6. The engineered, non-naturally occurring Type II Cas protein of claim 2, wherein the Cas protein comprises a set of mutations selected from: (a) A1113R and K1315Q; (b) L1298R and K1315Q; (c) A1113R and K1315T; (d) A1113R, L1298R and K1315Q; and (e) A1113R, L1298R and K1315T.

7. The engineered, non-naturally occurring Type II Cas protein of claim 1, wherein the Cas protein is capable of recognizing a protospacer adjacent motif (PAM) having a sequence selected from the group consisting of: NATACT, NATAGT, NATAAT and NATATT.

8. The engineered, non-naturally occurring Type II Cas protein of claim 7, wherein the Cas protein comprises an amino acid mutation at K1315, optionally, the mutation at position K1315 is K1315Q or K1315T.

9. The engineered, non-naturally occurring Type II Cas protein of claim 2, wherein the Cas protein comprises a mutation at at least one position selected from A1113, L1298, and K1315, and exhibits at least one of the following properties:
(a) increased activity compared to the wild-type Cas protein of SEQ ID NO: 69; and
(b) capability of recognizing a protospacer adjacent motif (PAM) having a sequence selected from the group consisting of NATACT, NATAGT, NATAAT, and NATATT;
optionally, the mutation at A1113, if present, is A1113R; the mutation at L1298, if present, is L1298R; and the mutation at K1315, if present, is K1315Q or K1315T.

10. The engineered, non-naturally occurring Type II Cas protein of claim 2, wherein the Cas protein comprises an amino acid sequence that has 100%, or at least 70%, at least 75%, at least 80%, at least 85%, at least 90%, at least 92%, at least 95%, at least 98%, or at least 99% sequence identity to any one of amino acid sequences of SEQ ID NOs: 81-95.

11. The engineered, non-naturally occurring Type II Cas protein of claim 1, wherein the Cas protein is a nickase or a dead Cas protein.

12. An engineered, non-naturally occurring CRISPR-Cas system comprising:
(a) an engineered, non-naturally occurring Type II Cas protein or a polynucleotide encoding the engineered, non-naturally occurring Type II Cas protein; and
(b) at least one engineered guide RNA (gRNA) or at least one engineered polynucleotide encoding the engineered gRNA, wherein said engineered gRNA comprises a spacer sequence that is complementary to a target nucleic acid and a Cas protein binding segment that interacts with said engineered, non-naturally occurring Type II Cas protein, wherein the Cas protein binding segment comprises a tracrRNA sequence and a direct repeat (DR) sequence, and wherein the tracrRNA sequence hybridizes with the DR sequence to form a double-stranded RNA (dsRNA) duplex;
wherein the engineered, non-naturally occurring Type II Cas protein comprises: (i) an amino acid sequence that has 100%, or at least 70%, at least 75%, at least 80%, at least 85%, at least 90%, at least 92%, at least 95%, at least 98%, or at least 99% sequence identity to an amino acid sequence of SEQ ID NO: 69; or (ii) an amino acid sequence that has 100%, or at least 70%, at least 75%, at least 80%, at least 85%, at least 90%, at least 92%, at least 95%, at least 98%, or at least 99% sequence identity to an amino acid sequence of SEQ ID NO: 69, with an exception of an amino acid “M” at position 1 of the amino acid sequence of SEQ ID NO: 69;
optionally, the Cas protein comprises an amino acid mutation at one or more of the following positions: A1113, L1298, K1315 and Q1191.

13. The engineered, non-naturally occurring CRISPR-Cas system of claim 12, wherein the engineered guide RNA comprises a linker sequence connecting the tracrRNA sequence and the DR sequence to form a sgRNA scaffold.

14. The engineered, non-naturally occurring CRISPR-Cas system of claim 12, wherein the tracrRNA sequence comprises a modification that enhance at least one property selected from an activity and a stability of the CRISPR-Cas system compared to a wild type tracrRNA of SEQ ID NO: 821; wherein the modification comprises: (i) one or more nucleotide mutations, insertions, deletions, or any combination thereof that reduce interaction between the spacer sequence and the tracrRNA sequence; or (ii) at least one stabilized hairpin secondary structure at a position that does not interfere with oligonucleotide binding or editing, wherein the stabilized hairpin comprises:
a contiguous stem that is 1 to 5 nucleotides longer than a corresponding stem in a wild-type tracrRNA of SEQ ID NO: 821; or
a contiguous stem comprising 1 to 5 more C-G base pairs than the corresponding stem in the wild-type tracrRNA of SEQ ID NO: 821.

15. The engineered, non-naturally occurring CRISPR-Cas system of claim 12, wherein the tracrRNA sequence comprises a sequence having at least 70%, 75%, 80%, 85%, 88%, 90%, 92%, 94%, 95%, 96%, 98%, 99% or 100% identity to any one of SEQ ID NOs: 821-826.

16. The engineered, non-naturally occurring CRISPR-Cas system of claim 13, wherein the sgRNA scaffold comprises a sequence having at least 70%, 75%, 80%, 85%, 88%, 90%, 92%, 94%, 95%, 96%, 98%, 99% or 100% identity to any one of SEQ ID NOs: 841-846.

17. The engineered, non-naturally occurring CRISPR-Cas system of claim 12, wherein the polynucleotide encoding the engineered, non-naturally occurring Type II Cas protein:
(a) is operably linked to a promoter selected from a constitutive promoter, a tissue-specific promoter, and an inducible promoter;
(b) is present in a vector selected from a retroviral vector, a lentiviral vector, a phage vector, an adenoviral vector, an adeno-associated virus vector, a herpes simplex virus vector and a plasmid vector; and
(c) is selected from a ribonucleotide sequence, a deoxyribonucleotide sequence, analogs thereof, and codon-optimized variants thereof for expression in a eukaryotic cell.

18. The engineered, non-naturally occurring polynucleotide of claim 12, wherein the engineered, non-naturally occurring polynucleotide is an mRNA, and further comprises a 5′ cap sequence and/or a poly-A tail sequence.

19. The engineered, non-naturally occurring CRISPR-Cas system of claim 12, wherein the polynucleotide encoding the engineered, non-naturally occurring Type II Cas protein has a sequence identity of at least 70%, 75%, 80%, 85%, 88%, 90%, 92%, 94%, 95%, 96%, 98%, 99%, or 100% to a nucleotide sequence of any one of SEQ ID NOs: 169, 181-195, 689, and 731-745.

20. A method of modifying a target DNA locus, wherein the method comprises delivering to said locus an engineered, non-naturally occurring Type II Cas protein, wherein the engineered, non-naturally occurring Type II Cas protein comprises:
(a) an amino acid sequence that has 100%, or at least 70%, at least 75%, at least 80%, at least 85%, at least 90%, at least 92%, at least 95%, at least 98%, or at least 99% sequence identity to an amino acid sequence of SEQ ID NO: 69; or
(b) an amino acid sequence that has 100%, or at least 70%, at least 75%, at least 80%, at least 85%, at least 90%, at least 92%, at least 95%, at least 98%, or at least 99% sequence identity to an amino acid sequence of SEQ ID NO: 69, with an exception of an amino acid “M” at position 1 of the amino acid sequence of SEQ ID NO: 69;
optionally, the Cas protein comprises an amino acid mutation at one or more of the following positions: A1113, L1298, K1315 and Q1191.
